(19) United States
(12) Patent Application Publication
(10) Pub. No.: US 2007/0233219 A1
(43) Pub. Date: Oct. 4, 2007

(54) Polymeric heart restraint

(76) Inventors: Bilal Shafi, Palo Alto, CA (US); Carlos Mery, Mountain View, CA (US); Qi Liao, Stanford, CA (US)

Correspondence Address: MORRISON & FOERSTER LLP, 755 PAGE MILL RD, PALO ALTO, CA, 94304-1018, US

(21) Appl. No.: 11/707,348

(22) Filed: Feb. 16, 2007

Related U.S. Application Data
(60) Provisional application No. 60/773,983, filed on Feb. 16, 2006.
(60) Provisional application No. 60/809,658, filed on May 30, 2006.

(51) Int. Cl. A61F 2/06; C08F 8/00

(52) U.S. Cl. 623/1.1; 528/354

(57) ABSTRACT

Described here are polymer compositions, methods, and systems for reinforcing a wall of a heart. The polymer compositions may be adapted to form networks, e.g., cross-linked networks, semi-interpenetrating networks, or interpenetrating networks, and placed within the pericardial space or on one or more pericardial tissues. The mechanical properties of the polymer compositions or networks derived therefrom may then be employed to reinforce a heart wall to prevent dilatation of a chamber of the heart and/or expansion of an infarct, e.g., to treat or prevent congestive or chronic heart failure.

### CROSS-REFERENCE TO RELATED APPLICATIONS

This application claims the benefit of U.S. Provisional Application Ser. No. 60/773,983, filed on Feb. 16, 2006, and U.S. Provisional Application Ser. No. 60/809,658, filed on May 30, 2006, each of which is hereby incorporated by reference in its entirety.

### FIELD

The compositions, methods, and systems described here relate to the field of cardiac physiology. More specifically, the compositions, methods, and systems relate to polymer networks that when placed on a pericardial tissue or within the pericardial space can function to reinforce a heart wall to prevent a chamber of the heart from dilating, induce reverse remodeling in a dilated heart, and/or reduce infarct expansion.

### BACKGROUND

Congestive heart failure (CHF) occurs when the heart is no longer able to pump enough oxygenated blood to the organs and tissues of the body to satisfy its metabolic needs. There are various etiologies of CHF, but typically all result in a dilated left ventricle with decreased contractility. The process of ventricular dilatation and remodeling (i.e., reshaping) is complex and poorly understood, but in low-output systolic heart failure, it is generally considered to be the result of chronic volume overload due to valvular dysfunction or ischemic damage to the myocardium. In order to maintain cardiac output in these states, some cardiac dilatation occurs to improve the short-term function of the heart, but the additional mechanical stress on the heart wall can lead to a vicious cycle of further injury and pathologic dilatation, and chronic heart failure.

Specifically, one of the effects of ventricular dilatation is a significant increase in wall stress due to cardiac wall thinning and increased radius, according to Laplace's law. This elevated wall stress serves to permanently injure already stunned myocytes, reversibly injure additional myocytes, and induce further cardiac dilatation. Furthermore, because cardiac output progressively diminishes as the degree of heart failure increases, end-diastolic volume within the ventricles continues to rise, resulting in continually rising wall stress levels. This increased diastolic wall stress is thought to be a major contributor to ongoing ventricular dilatation.

Symptoms of CHF generally develop when the remodeled left ventricle can no longer compensate for a poorly functioning heart. Individuals may experience fatigue and shortness of breath with varying degrees of activity, and in severe cases, may experience shortness of breath at rest or with very little exertion. Pulmonary and peripheral edema may also develop. Currently, approximately five million Americans suffer from some degree of heart failure, with 500,000 new cases being reported every year (Jessup, M., Heart Failure, The New England Journal of Medicine 348:2007-2018 (2003)).

Current treatment options to minimize ventricular remodeling are suboptimal. For example, medications such as beta-blockers and angiotensin-converting enzyme (ACE)-inhibitors are often prescribed to attenuate cardiac remodeling and improve survival after myocardial infarction. However, despite a risk reduction of 15%-40% (McMurray, J J. and M. A. Pfeffer, Heart Failure, Lancet 365:1877-1889 (2005)), most patients continue their progression to congestive heart failure, albeit at a slower rate (Gheorghiade M. and R. O. Bonow, Chronic Heart Failure in the United States: A Manifestation of Coronary Artery Disease, Circulation 97:282-289 (1998)). Furthermore, increasing dosages of these medications are generally associated with increased side-effects.

Another treatment option involves surgical placement of a passive restraint around the heart after an acute myocardial infarction to alleviate wall stress and thereby prevent infarct expansion and subsequent cardiac remodeling (Magovern J. A. et al., Effect of a Flexible Ventricular Restraint Device on Cardiac Remodeling After Acute Myocardial Infarction, Asaio J 52:196-200 (2006)). Examples of devices currently in development include the CorCap™ cardiac support device (Acorn Cardiovascular, St. Paul, Minn.), the Myosplint™ device (Myocor, Inc., Maple Grove, Minn.), and the Paracor cardiac support device (Paracor Medical, Inc., Sunnyvale, Calif.). However, these devices have been primarily indicated for use in treating end-stage CHF. In addition, placement of these devices can be highly invasive (e.g., requiring open chest surgery). Further, any future surgical procedures performed to the heart can be difficult due to the development of fibrous tissue adhesions around the device and structures surrounding the heart. Animal studies with 6-12 weeks of follow-up have demonstrated that placement of a passive restraint device can support the heart after an infarction and alleviate wall stress to prevent infarct expansion, ventricular dilation, cardiac remodeling, wall thinning, and progression to CHF. One effect of the restraint devices appears to be preventing remodeling processes in the heart. Recently, U.S. Patent publication no. 2006/0229492 has disclosed methods and systems to constrain a heart using polymeric compositions injected into the pericardial space.

Accordingly, it would be desirable to have compositions, methods and systems to reinforce a heart wall to prevent a chamber of the heart from dilating, where such compositions, methods and systems can be delivered using minimally invasive procedures. It would also be desirable to have compositions, systems, and methods that are capable of preventing cardiac remodeling and/or infarct expansion. Similarly, it would be desirable to have compositions, systems, and methods that employ their reinforcing function to treat or prevent congestive heart failure or chronic heart failure.

### SUMMARY

Described herein are polymer compositions, methods, and systems that can be used to reinforce a heart wall to prevent or reverse dilatation of the heart, or to prevent or reduce infarct expansion, e.g., to treat congestive or chronic heart failure or to prevent the development of congestive or chronic heart failure after a myocardial infarction. A polymeric matrix derived from the polymer compositions can be disposed as single layer or multilayer film or shell on or between one or more pericardial tissues, e.g., in the pericardial space. The polymeric matrix can be designed to have one or more properties (e.g., elasticity and/or tensile strength) that allow the film or shell formed from the matrix to reinforce at least a portion of a myocardial wall to prevent heart chamber dilatation or infarct expansion, while still allowing the heart to fill properly. Further, a polymeric matrix derived from the polymer compositions described herein can be used as permanent or temporary myocardial wall reinforcement, e.g., a polymeric matrix that degrades over time at a desired rate can be used. Biodegradable or nonbiodegradable polymers may be employed, so long as they are biocompatible.

In some variations, polymeric compositions are provided. The compositions comprise a triblock copolymer having the formula (CL)ₙ-(EG)ₘ-(CL)ₗ, where CL is a caprolactone monomeric unit, EG is an ethylene glycol monomeric unit, l and n are integers from 1 to 18, and m is an integer from 70 to 400. The triblock copolymer is functionalized with at least one cross-linkable group, e.g., an acrylate, an amine, a sulfhydril, or N-hydroxysuccinimide. In some variations, the triblock copolymer can be terminated with a cross-linkable acrylate group. For example, in some compositions l can be 2 or 3 or 4 and n can be 2 or 3 or 4. In still other compositions, m can be 130 to 200.

The compositions can be cross-linked to form at least a portion of a polymeric matrix. In some variations, the polymeric matrix may comprise a first network derived from the triblock copolymer. For example, the triblock copolymer can be functionalized with an amine, and the polymeric matrix can be at least partially formed by cross-linking the functionalized triblock copolymer with a poly(ethylene glycol) functionalized with N-hydroxysuccinimide. The polymeric matrix may comprise a semi-interpenetrating network comprising a second polymer infused or entangled into a first cross-linked network derived from the triblock copolymer. The second polymer can be selected from the group consisting of alginate, casein, chitin, collagen, gelatin, hyaluronic acid, poly(ethylene glycol) and derivatives thereof, poly(caprolactone) and derivatives thereof, blends thereof, and copolymers thereof. For example, the second polymer in the semi-interpenetrating network can be collagen. In still other variations, the polymeric matrix can comprise an interpenetrating network comprising a first cross-linked network derived from the triblock copolymer and a second cross-linked network derived from a second polymer. Here, also, the second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof. For example, the second polymer in the interpenetrating network can be cross-linked collagen.

The number of CL monomeric units (n, l) and the number of EG monomeric units (m) may be varied to adjust physical and/or mechanical properties of a polymeric matrix derived from the composition. For example, the polymeric matrix can have an in vivo elastic modulus of about 200 kPa to about 1.5 MPa or even higher, e.g., about 700 kPa, about 900 kPa, or about 1 MPa at strains above about 20%. The polymeric matrix can have an in vivo ultimate tensile strength of about 200 kPa or greater. Some polymeric matrices can have a combination of mechanical properties, e.g., in some variations the matrices can have an in vivo elastic modulus of 200 kPa or greater at strains above about 20% and an ultimate tensile strength of about 200 kPa or greater. The in vivo degradation rate of polymeric matrices derived from the compositions can also be tuned, e.g., to vary the length of a treatment using the polymeric matrices. For example, an in vivo elastic modulus of a polymeric matrix derived from the compositions can decrease at an average rate of about 0.01% to about 1% per day. The compositions and/ or polymeric matrices derived from the compositions can be adapted to be delivered by injection, e.g., as a powder or in a fluid form, e.g., as a liquid, gel, suspension, or solution.

Methods for reinforcing at least a portion of a wall of a heart chamber are provided. The methods include accessing a pericardial tissue or a pericardial space and applying a sufficient amount of a polymeric matrix to the pericardial tissue or pericardial space to prevent dilatation of the heart chamber or expansion of an infarct. The polymeric matrix is derived from a triblock copolymer having the formula (CL)ₗ-(EG)ₘ-(CL)ₙ where CL is a caprolactone monomeric unit, EG is an ethylene glycol monomeric unit, and l, m, and n are integers. For example, in some variations, the methods can include functionalizing the triblock copolymer with an amine, and at least partially forming the polymeric matrix by cross-linking the functionalized triblock copolymer with a poly(ethylene glycol) that is functionalized with N-hydroxysuccinimide. The methods can be used for treating or preventing congestive or chronic heart failure. For example, the methods can be used for preventing dilatation of a heart chamber or preventing expansion of an infarct.

In some variations, the methods can include applying the polymeric matrix to the fibrous pericardium, the parietal pericardium, or the visceral pericardium. The methods can be used to prevent dilatation of the left ventricle. In the methods described herein, the pericardial tissue or pericardial space can be accessed using any suitable technique. For example, image guidance can be used in some variations. In other methods, thoracoscopy can be used, or any combination of the aforementioned methods. The methods can include accessing the pericardial tissue or the pericardial space via a heart chamber or via an atrial wall.

In some variations, the methods can include percutaneously inserting a conduit to access the pericardial space or the pericardial tissue. In these variations, the polymeric matrix or a precursor form of the polymeric matrix can be applied to the pericardial tissue of pericardial space via the conduit. For example, the conduit can be inserted through a femoral vessel or through a transpericardial opening. When a transpericardial or a transmyocardial opening is made, the opening can be sealed with the polymeric matrix.

In some methods, at least a portion of the polymeric matrix can be formed prior to application. In other methods, at least a portion of the polymeric matrix can be formed during or after delivering the triblock copolymer to the pericardial tissue or the pericardial space. For example, UV irradiation of the triblock copolymer can be used to form at least a portion of the polymeric matrix after the triblock copolymer has been delivered to the pericardial tissue or the pericardial space.

Methods can include applying the polymeric matrix to the pericardial tissue or space by delivering the polymeric matrix or a precursor to the polymeric matrix to the pericardial tissue or space as a powder or as a fluid, e.g., as a liquid, gel, suspension, or solution. Then the powder or fluid can be processed in situ to form a solid film. For example, the fluid can be cured in situ to form the solid film by cross-linking, gelation, heating and/or drying. The fluid or powder can also bind (e.g., cross-link) to the surface of the heart.

Some variations of the methods can include applying a polymeric matrix comprising a semi-interpenetrating network to a pericardial tissue or a pericardial space. The semi-interpenetrating network can comprise a first cross-linked network of the triblock copolymer, where the first cross-linked network is entangled or infused with a second polymer. In these variations, the methods can include providing a solution of the triblock copolymer and the second polymer, and cross-linking the triblock copolymer in the solution to form a first cross-linked network in the presence of the second polymer to form the semi-interpenetrating network. The second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof. For example, in some of these variations, the second polymer is collagen.

Other variations of the methods include applying a polymeric matrix comprising an interpenetrating network to the pericardial tissue or pericardial space. The interpenetrating network can comprise a first cross-linked network derived from the triblock copolymer interpenetrated with a second cross-linked network derived from a second polymer. In these variations, for example, the methods can include providing a solution of the triblock copolymer and the second polymer and cross-linking one of the triblock copolymer and the second polymer in the solution to form the first network, and subsequently cross-linking the other to form the second network, thereby forming the interpenetrating network. The second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof. For example, the second polymer of the interpenetrating network can be collagen.

Still other variations of the methods can include applying a polymeric matrix comprising a super polymer network to the pericardial tissue or pericardial space. The super polymer network can comprise the triblock copolymer cross-linked with a second polymer. In these variations, for example, the methods can include providing a solution of the triblock copolymer and the second polymer, and cross-linking both polymers simultaneously to form a super polymer network in which the triblock copolymer will be cross-linked to other triblock copolymers as well as to the second polymer. The second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof.

The physical and/or mechanical properties of the polymeric matrix applied as part of the methods can be tuned to adjust the ability of a film of the polymeric matrix to reinforce a heart wall. In particular, the polymeric matrix can be selected to have a nonlinear elastic modulus characterized as having an in vivo elastic modulus at strains below about 20% that is lower than the in vivo elastic modulus at strains above about 20% strain. In some variations, the polymeric matrix can have an in vivo elastic modulus of about 200 kPa or greater at strains above about 20%. The polymeric matrix can have an in vivo ultimate tensile strength of about 200 kPa or greater. Some polymeric matrices can have a combination of mechanical properties, e.g., in some variations the matrices can have an in vivo elastic modulus of about 200 kPa or greater at strains above about 20% and an ultimate tensile strength of about 200 kPa or greater. The in vivo degradation rate of polymeric matrices derived from the compositions can also be tuned, e.g., to vary the length of time the polymeric matrix operates to prevent dilatation of a heart chamber and/or expansion of an infarct. For example, an elastic modulus of a polymeric matrix derived from the compositions can decrease at an average rate of about 0.01% to about 1% per day.

In some methods, the polymeric matrix is capable of reinforcing a myocardial wall for at least 2 months. In other methods, the polymeric matrix is capable of reinforcing the myocardial wall for at least 4 months. In still other variations of the methods, the polymeric matrix is capable of reinforcing the myocardial wall for at least 6 months, or even longer, e.g., indefinitely.

Additional methods for reinforcing at least a portion of a wall of a heart chamber are provided. These methods include accessing a pericardial tissue, applying a first layer of a first polymer to the pericardial tissue, and adhering a second layer of a second polymer to the first layer to form a polymeric matrix in sufficient amount to prevent dilatation of the heart chamber and/or expansion of an infarct. In these methods, the tissue can include the fibrous pericardium, the parietal pericardium, and the visceral pericardium. The methods can include percutaneously inserting a conduit to access the pericardial tissue, delivering the first polymer to the tissue via the conduit and forming the first layer, and delivering the second polymer via the conduit to the first layer.

In some variations of these methods, the first polymer can comprise any suitable primer layer, e.g., a layer that can mechanically or chemically bind with the tissue. The second polymer can comprise a triblock copolymer having the formula (CL)ₗ-(EG)ₘ-(CL)ₙ, where CL is a caprolactone monomeric unit, EG is an ethylene glycol monomeric unit, and l, m, and n are integers. For example, l and n can be 1 to 18 and m can be 20 to 400. The second polymer layer can comprise a semi-interpenetrating network or an interpenetrating network comprising the second polymer and a third polymer. The third polymer can be selected from the group consisting of alginate, chitosan, casein, chitin, collagen, gelatin, hyaluronic acid, poly(ethylene glycol) and derivatives thereof, poly(caprolactone) and derivatives thereof, blends thereof, and copolymers thereof. For example, the third polymer can be collagen.

In these methods, the second polymer layer can be adhered to the first polymer layer by a cross-linking reaction between the first and second polymers. In some variations, the first polymer layer can be applied to the pericardial tissue by delivering the first polymer or a precursor of the first polymer as a powder or as a fluid, e.g., as a liquid, gel, suspension or solution, to the pericardial tissue and processing the fluid or powder in situ to form a solid film. For example, the fluid can be cured by cross-linking, heating, gelation, and/or drying to form the solid film comprising the first polymer. The second layer can be adhered to the first layer by delivering the second polymer or a precursor of the second polymer as a powder or fluid to the first layer, and processing the fluid or powder in situ to form a solid laminate comprising the first and second polymers. For example, a fluid comprising the second polymer can be cured by cross-linking, gelation, heating, and/or drying to form the solid laminate.

Systems for reinforcing at least a portion of a wall of a heart chamber are also provided. The systems comprise a polymer composition adapted to form a polymeric matrix, the composition comprising a triblock copolymer having the formula (CL)ₗ-(EG)ₘ-(CL)ₙ. CL is a caprolactone monomeric unit, EG is an ethylene glycol monomeric unit, and l, m, and n are integers. The systems comprise a first conduit configured to access a pericardial space or a pericardial tissue and to deliver the polymer composition or the polymeric matrix to the pericardial space or tissue. In some variations, the first conduit comprises a balloon that is configured to deliver the polymer composition or the polymeric matrix to the pericardial space or tissue.

Some variations of the systems can comprise a second polymer, e.g., a second polymer selected from the group consisting of alginate, casein, chitin, chitosan, collagen, gelatin, hyaluronic acid, poly(ethylene glycol) and derivatives thereof, poly(caprolactone) and derivatives thereof, blends thereof, and copolymers thereof. For example, some systems can include collagen as a second polymer. In these systems, the triblock copolymer can be adapted to form a polymeric matrix comprising a semi-interpenetrating network comprising a cross-linked network of the triblock copolymer infused or entangled with the second polymer. In other systems including a second polymer, the triblock copolymer can be adapted to form a polymeric matrix comprising an interpenetrating network comprising a first cross-linked network derived from the triblock copolymer, and a second cross-linked network derived from the second polymer. Systems comprising a second polymer can include a second conduit configured to access the pericardial space or the pericardial tissue and to deliver the second polymer to the pericardial tissue or pericardial space.

Some variations of the systems include an initiator to cause formation of at least a portion of the polymeric matrix. For example, the initiator can cause the creation of free radicals. Initiators can be selected from the group consisting of a light source, a radiation source, a solution having a desired pH or ionic concentration, a chemical cross-linking agent, heat, and combinations thereof. For example, in some systems, the initiator can comprise a UV light source.

### BRIEF DESCRIPTION OF THE DRAWINGS

FIG. 1A shows a cross-sectional view of a normal human heart in diastole.

FIG. 1B shows a cross-sectional view of a diseased human heart in diastole, where the heart exhibits dilatation of the left ventricle.

FIG. 2 depicts a simplified cross-sectional view of the pericardial tissues and spaces that surround the heart.

FIG. 3 illustrates how an exemplary polymeric matrix applied to the pericardial space can constrain the heart.

FIGS. 4A-4C illustrate schematic diagrams of a single polymeric network, a semi-interpenetrating network, and an interpenetrated network, respectively.

FIG. 5 depicts the structure of an exemplary A-B-A triblock copolymer, where A is poly(caprolactone) and B is poly(ethylene glycol). The triblock copolymer is end-capped with acrylate groups in this example.

FIG. 6 illustrates an exemplary method of applying a polymeric matrix to the pericardial space.

FIGS. 7A-B illustrate an exemplary polymeric matrix applied to the exterior surface of a heart to reinforce a portion of a wall of the heart. FIG. 7B shows a cross-sectional view taken along line I-I′ in FIG. 7A.

FIG. 8 illustrates examples of stress-strain curves of thin films made from a polymeric matrix that can be used to reinforce a heart wall.

### DETAILED DESCRIPTION

Described herein are compositions, methods, and systems for reinforcing a myocardial wall to prevent or reverse dilatation of the heart, e.g., by inducing reverse remodeling, and/or prevent infarct expansion. In general, the compositions are polymer compositions that may form matrices that can be disposed on or between pericardial tissues. “Infarct expansion” and “infarct extension” refer to thinning or stretching of tissue in and around an infarct zone that extends to an area outside the initial infarct zone. As used herein, the term “prevent” or “preventing” refers to the complete avoidance of heart dilatation or infarct expansion, halting of further heart dilatation or infarct expansion, slowing of heart dilatation or infarct expansion, reversing heart dilatation or infarct expansion, or improvement in heart dilatation or infarct expansion. Furthermore, as used herein, the terms “dilatation,” “dilation,” and “dilate,” shall be used interchangeably, and refer to any enlargement or expansion of one or more chambers of the heart outside of normal. As used herein, the term “passive” refers to a device, system, or method in which external power or activation is not necessary for ongoing operation. Thus, a passive heart restraint is one that can operate to reinforce the heart without external power or activation after delivery and installation.

The term “polymer network” as used herein encompasses polymer compositions with interchain interactions (cross-links) to form a three-dimensional network. The degree of cross-linking can vary between polymer networks, and can be used to tune chemical, physical, and/or mechanical properties of a polymer network. The cross-linking interactions between polymer chains in a polymer network can be covalent or non-covalent. Any suitable method can be used to form covalent bonds between polymer chains to result in a polymer network, e.g., use of chemical cross-linking agents, photo-initiated cross-linking, radiation-induced cross-linking (e.g., electron beam), thermally-induced cross-linking, or combinations thereof. Examples of non-covalent cross-linking mechanisms include hydrogen bonding, hydrophilic or hydrophobic interactions, and ionic or electrostatic interactions.

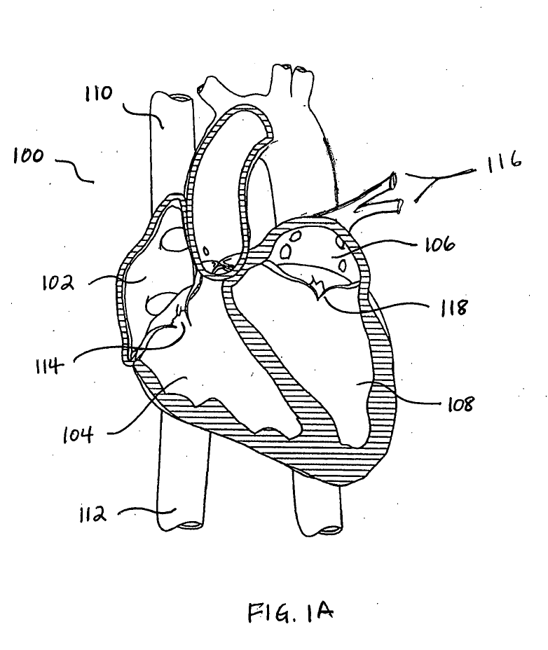

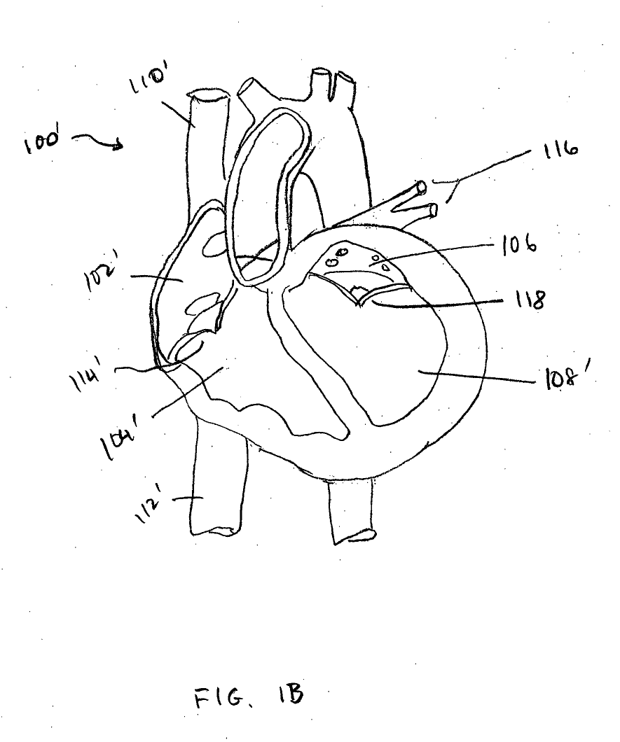

Referring now to FIG. 1A, a healthy heart is depicted in diastole. The heart 100 includes four chambers, the right atrium (RA) 102, the right ventricle (RV) 104, the left atrium (LA) 106, and the left ventricle (LV) 108. Deoxygenated blood returns to the RA 102 via the superior vena cava 110 and inferior vena cava 112, and enters the RV 104 through the tricuspid valve 114. The RV 104 then pumps the deoxygenated blood to the lungs (not shown) via the pulmonary artery (not shown). Oxygenated blood returns to the LA 106 via the pulmonary veins 116, and then travels into the LV 108 through the mitral valve 118. Upon contraction of the LV 108, oxygenated blood enters the systemic circulation.

In FIG. 1B, a diseased heart in diastole that shows evidence of dilatation of the left ventricle is illustrated. Diseased heart 100′ has right atrium 102′, right ventricle 104′, left atrium 106′, left ventricle 108′, superior vena cava 110′ and inferior vena cava 112′. Left ventricle 108′ is enlarged and distorted. The distortion of one or more chambers of the heart can also lead to valve regurgitation.

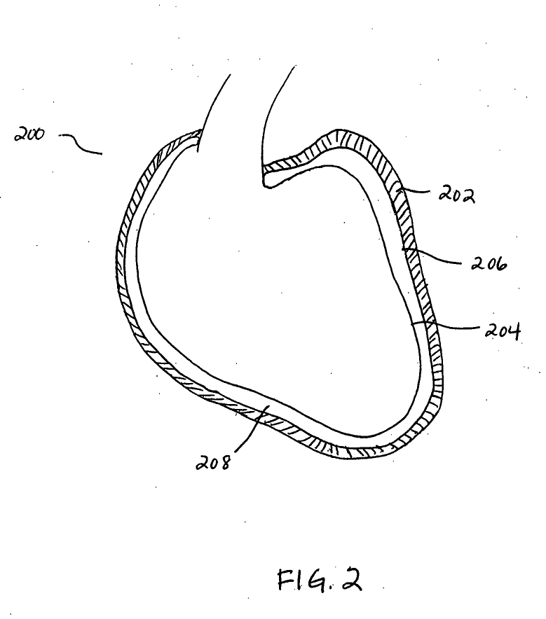

As shown in FIG. 2, the heart 200 is enclosed in a double-walled fibroserous sac called the pericardium. The pericardium consists of two parts: 1) a strong external layer composed of tough, fibrous tissue, called the fibrous pericardium 202; and 2) an internal double-layered sac composed of a transparent membrane called the serous pericardium. The layer of serous pericardium that is reflected onto the surface of the heart is known as the visceral pericardium 204, and forms the epicardium (external layer of the heart wall). The layer of serous pericardium that is fused to the fibrous pericardium 202 is known as the parietal pericardium 206. The space between the parietal pericardium 206 and visceral pericardium 204 is referred to as the pericardial space 208.

Compositions

The polymer compositions provided herein and the polymeric matrices derived therefrom may be adapted for placement on any pericardial tissue or in the pericardial space. As stated above, the polymeric matrices can form a film or shell that supports at least a portion of a myocardial wall to prevent dilatation of a chamber and/or expansion of an infarct surrounded by that wall. Thus, in some variations, the polymer compositions can be adapted for placement on the fibrous pericardium. In other variations, the polymer compositions can be adapted for placement on the parietal pericardium. In yet other variations, the polymer compositions can be adapted for placement on the visceral pericardium. In further variations, the polymer compositions can be adapted for placement in the pericardial space. The polymer compositions can also be placed in more than one of these locations, e.g., in the pericardial space as well as on the parietal pericardium and/or on the visceral pericardium and/or on the fibrous pericardium. More than one polymer composition may be applied to the same heart. For example, one polymer composition can be placed in the pericardial space, whereas a different polymer composition can be placed on the parietal pericardium, on the visceral pericardium, or on the fibrous pericardium.

The polymer compositions are adapted to form a polymeric matrix that includes at least one polymer network, e.g., a cross-linked polymer network, a semi-interpenetrating network, or an interpenetrating network. Thus, a polymeric matrix derived from the polymer composition can be adapted for placement on the fibrous pericardium, the parietal pericardium, visceral pericardium, or in the pericardial space. The polymeric matrix can also be placed in more than one of these locations, e.g., in the pericardial space as well as on parietal pericardium and/or on the visceral pericardium and/or on the fibrous pericardium. More than one polymeric matrix may be applied to the same heart. For example, one polymeric matrix can be placed in the pericardial space, whereas a different polymeric matrix can be placed on the parietal pericardium, visceral pericardium, or the pericardium tissue. Further, a polymer matrix may comprise more than one layer, e.g., a polymer matrix may be a laminate of two or more polymeric layers.

A polymeric matrix has at least one property, for example, elasticity and/or tensile strength, such that when the polymeric matrix surrounds at least a portion of a heart chamber, e.g., as a film or a shell, the matrix can function to prevent the heart chamber from dilating further with time or an infarct from expanding, while still allowing the heart to fill. For example, the polymeric matrix can have sufficient elasticity to allow proper diastolic filling of the heart, but also have an elastic limit that can prevent the heart from expanding beyond a desired volume. Specifically, by placing or forming a film or shell comprising the polymeric matrix around at least a portion of the heart, the particular property of the polymeric matrix (e.g., elastic limit) can allow the film or shell to relieve stress on the myocardial wall by limiting end-diastolic volume. This reduction in myocardial wall stress (afterload) reduces the amount of work the heart must perform. In turn, the risk of further myocardial damage is decreased by the reduction in myocardial oxygen and metabolic requirements.

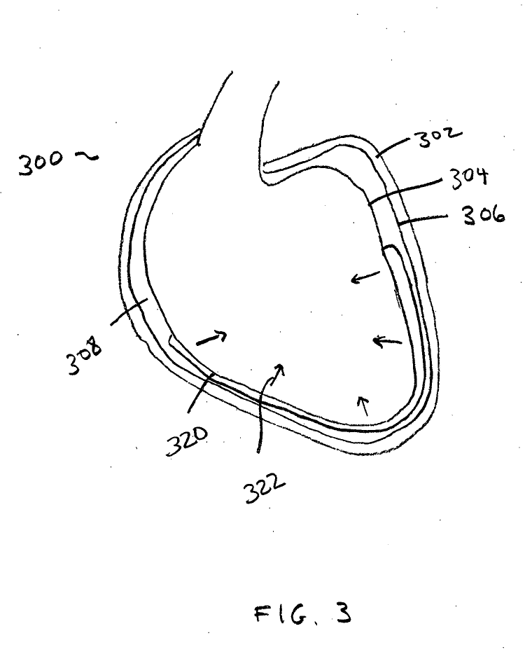

For example, as shown in FIG. 3, a polymeric matrix derived from a polymer composition as herein described is disposed as a film in the pericardial space of a heart. Diseased heart 300 has fibrous pericardium 302, visceral pericardium 304, parietal pericardium 306, and pericardial space 308. Film 320 made from the polymeric matrix may provide mechanical support in the direction of the arrows 322 to reduce myocardial wall stress by restraining at least a portion of the heart, e.g., the left ventricle, from further dilatation. The polymeric matrix film may be designed as a temporary or permanent myocardial wall reinforcement, e.g., to provide a desired level of mechanical support for a desired length of time.

Any biocompatible polymer, biodegradable or non-biodegradable, may be included in the compositions and matrices derived therefrom used to support a heart wall. The selection of the biodegradable or nonbiodegradable polymer to be employed can vary depending on the elasticity, tensile strength, residence time desired (e.g., as determined by degradation rate), method of delivery, and the like. In all instances, the biodegradable polymer when degraded results in physiologically acceptable degradation products.

Exemplary biocompatible and biodegradable polymers include alginate, casein, chitin, chitosan, collagen, gelatin, gluten, hyaluronic acid, a poly(lactide); a poly(glycolide); a poly(lactide-co-glycolide); a poly(lactic acid); a poly(glycolic acid); a poly(lactic acid-co-glycolic acid); poly(lactide)/poly(ethylene glycol) copolymers; poly(glycolide)/poly(ethylene glycol) copolymers; poly(lactide-co-glycolide)/poly(ethylene glycol) copolymers; poly(lactic acid)/poly(ethylene glycol) copolymers; poly(glycolic acid)/poly(ethylene glycol) copolymers; poly(lactic acid-co-glycolic acid)/poly(ethylene glycol) copolymers; a poly(caprolactone); poly(caprolactone)/poly(ethylene glycol) copolymers; a poly(orthoester); a poly(phosphazene); a poly(hydroxybutyrate); a poly(lactide-co-caprolactone); a polycarbonate; a polyesteramide; a polyanhydride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oxyethylene)/poly(oxypropylene) copolymer; and copolymers and blends thereof.

If a nonbiodegradable polymer is used in the composition, suitable nonbiodegradable polymers include poly(ethylene vinyl acetate), poly(vinyl acetate), silicone, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, poly(vinyl alcohol), polyphosphazene, poly(hydroxyalkanoate), poly(vinyl pyrrolidone), poly(hydroxyl methacrylate), ethylene glycol-butylene terephthalate copolymer, ethylene-vinyl acetate copolymer, polyesters, polyurethanes, polycarbonates, and copolymers and blends thereof.

The polymeric matrices may be of any form, so long as they possess one or more desired properties, e.g., an elasticity that allows proper filling and emptying of the heart, but which also prevents further dilatation of the heart and/or infarct expansion, e.g., an elastic limit. In general, the polymer compositions are cross-linked to form a first polymeric network. Any suitable cross-linking method can be used, e.g., photo-initiated cross-linking, radiation, e.g., electron beam cross-linking, thermally-induced cross-linking, or chemical cross-linking, e.g., by the addition of a chemical cross-linking agent. In some variations, the polymer compositions can be cross-linked in situ to form the polymeric matrix. In other variations, the polymer compositions can be at least partially cross-linked prior to delivery. In some cases, the polymer compositions can be cross-linked both before and after delivery. In still other variations, a primer layer can be first introduced that can mechanically or chemically bond with contacted pericardial tissue. The subsequently delivered polymer composition can bind with the primer layer and cross-link to provide the polymeric matrix around the heart to support the heart wall.

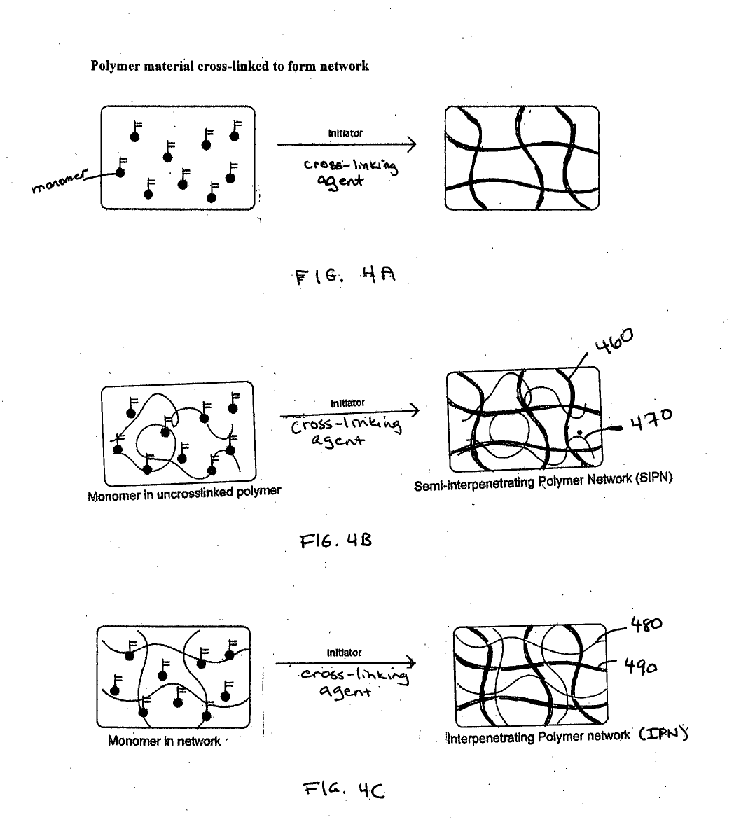

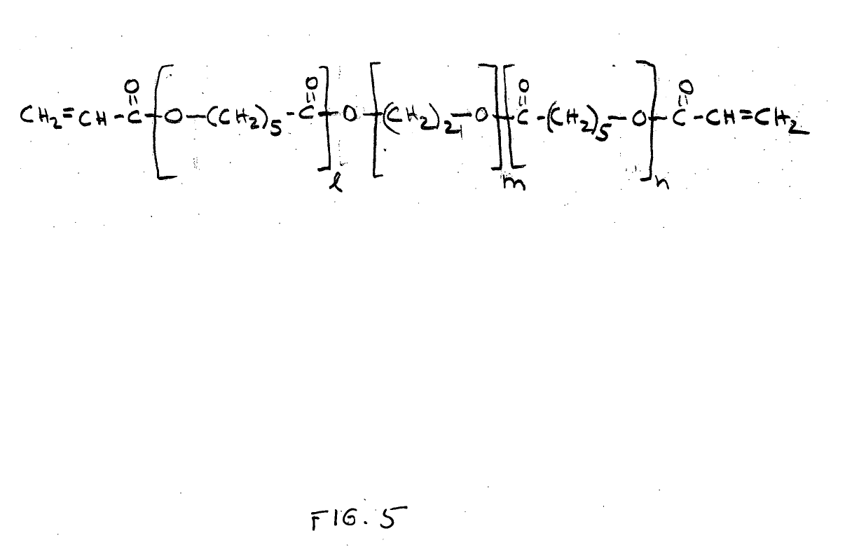

The polymeric matrix can include one or more semi-interpenetrating polymer networks, or one or more interpenetrating polymer networks, as defined by the IUPAC Compendium of Chemical Terminology, Electronic Version (goldbook.iupac.org/index.html). Semi-interpenetrating networks comprise one or more polymeric networks and one or more linear or branched polymers that infuse at least one of the polymeric networks on a molecular scale. Semi-interpenetrating networks may be considered polymer blends because the linear or branched polymer can be separated from the polymer network without breaking chemical bonds. A semi-interpenetrating network can achieve enhanced material properties (e.g., strength and/or elasticity) by the mere entanglement of the second uncrosslinked polymer within the first cross-linked network. A schematic showing a single polymeric network is provided in FIG. 4A. A schematic depicting a semi-interpenetrated network is provided in FIG. 4B, with linear or branched uncross-linked polymer 470 entangled with network 460 (indicated by bold lines).

An interpenetrating polymer network comprises two or more networks that are at least partially interlaced on a molecular scale but not covalently bonded to each other. In an interpenetrating network, the two networks cannot be separated unless chemical bonds are broken. Thus, a mixture of two or more preformed polymer networks is not an interpenetrating network. An interpenetrating polymer network can be formed by infusing a first polymeric network with a prepolymer (e.g., a monomer or oligomer) of a second polymer and cross-linking the prepolymer or the second polymer in the presence of the first polymeric network to form a second polymeric network interpenetrated with the first polymeric network. An interpenetrating polymer network can also be formed by mixing a first polymer or first polymer precursor (e.g., a monomer or oligomer) with a second polymer or second polymer precursor (e.g., a monomer or oligomer) and allowing each polymer to polymerize and/or cross-link to create two distinct, interlaced polymer networks. That is, the components of the mixture can be selected such that the first polymer chains and first polymer precursors do not polymerize or form cross-links to any significant degree with the second polymer chains or second polymer precursors. An interpenetrating polymer network can also be formed by infusing a second polymer into a first cross-linked network by swelling the first network with a solution of the second polymer and then cross-linking the second polymer to form the second network. A schematic showing an interpenetrated network is provided in FIG. 4C, where first network 480 is interlaced with second network 490 (indicated by bold lines). A super polymer network can comprise a combination of a first polymer cross-linked with a second polymer to create a single network.

In some variations, the polymeric matrix can comprise a hydrogel. The term “hydrogel” is meant to encompass a cross-linked polymer network that can be swollen with water or aqueous solution to form a gel. Although hydrogels can be swollen with water, they are not generally soluble in water. A hydrogel can absorb many times its weight in water, e.g., about 5 times, about 10 times, or about 50 times, or about 100 times or more than its weight in water.

In some variations, the polymer composition used to form the polymeric matrix to prevent heart dilatation and/or infarct expansion is a block copolymer, e.g., an A-B diblock copolymer or an A-B-A triblock copolymer. At least one of the A and B blocks can be selected to be biodegradable and determine an in vivo degradation rate, whereas the other of A and B blocks can be selected to impart strength (e.g., tensile strength) and/or elasticity to a matrix formed from the block copolymer. In addition, A and B can be selected to impart any desired physical or chemical property to a block copolymer or to a cross-linked matrix derived therefrom, e.g., solubility, viscosity, or a melt or glass transition temperature.

The diblock or triblock copolymers used in the compositions can be functionalized with one or more cross-linkable groups. In some variations of the compositions described herein, an A-B or A-B-A block copolymer may comprise a group that can be cross-linked upon exposure to UV light in the wavelength range from about 200 nm to about 400 nm. For example, the block copolymer may be functionalized with an acrylate group that can form cross-links upon irradiation with UV light. In other variations, the cross-linkable group may be capable of interacting with a cross-linking agent to form a cross-linked network from the block copolymers. For example, an amine- or sulfhydril-terminated A-B or A-B-A block copolymer can be cross-linked with the addition of a substance functionalized with N-hydroxysuccinimide (NHS), e.g., an NHS-functionalized ester.

In A-B diblock or A-B-A triblock copolymers useful in the compositions described herein, A and B can be selected to be a poly(caprolactone) (PCL), a poly(ethylene glycol) (PEG), a poly(lactic acid) (PLA), a polyfumarate, or a poly(lactic-co-glycolic acid) (PLGA). Thus, suitable compositions can include the following diblock copolymers: PCL-PEG; PLA-PEG; polyfumarate-PEG; and PLGA-PEG. Suitable compositions can include the following triblock copolymers: PCL-PEG-PCL; PLA-PEG-PLA; polyfumarate-PEG-polyfumarate; PLGA-PEG-PLGA; PEG-PCL-PEG; PEG-PLA-PEG; and PEG-polyfumarate-PEG.

For example, compositions can include an A-B-A triblock copolymer having the general formula (CL)ₗ-(EG)ₘ-(CL)ₙ, where CL is a caprolactone monomeric unit, EG is an ethylene glycol monomeric unit, and l, m, and n represent integers. (CL)ₗ-(EG)ₘ-(CL)ₙ will also be referred to as a PCL-PEG-PCL triblock copolymer herein. In some variations, (CL)ₗ-(EG)ₘ-(CL)ₙ triblock copolymers can be functionalized with one or more cross-linkable groups to facilitate the formation of a cross-linked matrix from the triblock copolymer. Suitable cross-linkable groups include an acrylate, an amine, or a sulfhydril. For example, the (CL)ₗ-(EG)ₘ-(CL)ₙ triblock copolymer can be end-capped with one or more acrylate groups, e.g., an acrylate unit on each end, as illustrated in FIG. 5 and described in Example 1. This diacrylated PCL-PEG-PCL triblock copolymer (denoted PCL-PEG-PCL-DA) may be cross-linked to form a polymer network as described below in Example 2.

The PCL and PEG blocks of the triblock copolymer may be varied to impart desired properties to the polymer composition before and/or after cross-linking. For example, the total number of CL monomeric units (i.e., l and n) included in the triblock copolymer may be varied, depending on desired factors such as solubility in water of the triblock copolymer before cross-linking, the viscosity of a corresponding polymer solution before or during delivery, and elasticity, tensile strength, and degradation rate of a cross-linked network derived from the triblock copolymer. In some variations of the (CL)ₗ-(EG)ₘ-(CL)ₙ triblock copolymer, n and l can each be from 1 to 18, or from 2 to 12, or from 2 to 7, or from 2 to 3. In other variations, 4 to 8 total CL monomeric units can be included in the triblock copolymer. In some cases, the sum of n and l is 7. In still other variations, n and l are each 2-3, resulting in a total of 4 to 6 monomeric CL units in the triblock copolymer. The size of the PCL blocks can also be used to adjust degradation rate, e.g., degradation rate can be increased by increasing n and l. Similarly, the number of EG monomeric units (m), and hence the molecular weight, of the PEG block in the triblock copolymer may be varied. For example, the molecular weight of the PEG block can be selected to provide a desired combination of elasticity and tensile strength in a polymeric matrix that is applied to the heart. In some variations, the PEG block of the triblock copolymer has a molecular weight about 1000 Da to about 15000 Da, or about 3000 Da to about 12000 Da, or about 5000 Da to about 10000 Da, or about 6000 Da to about 8000 Da, or about 6000 Da.

The compositions can be cross-linked to form at least a portion of a polymeric matrix that can be used to reinforce a myocardial wall. Such a polymeric matrix can comprise a single cross-linked polymer network derived from the PCL-PEG-PCL triblock copolymer. As discussed above and in Examples 1 and 2, such a cross-linked network can be formed by functionalizing the PCL-PEG-PCL with a cross-linkable group and then cross-linking by any suitable method. PCL-PEG-PCL can be amine-terminated or sulfhydril-terminated to allow cross-linking with a two-, three-, or four-arm NHS-functionalized substance used as a cross-linking agent. Further, a cross-linked PCL-PEG-PCL network can be formed by mixing in solution an amine-terminated or sulfhydril-terminated PCL with PEG terminated with NHS at 3 or 4 positions, or by mixing in solution an amine-terminated PEG (terminated at 3 or 4 positions) with NHS-terminated PCL. In other variations, a polymer matrix comprising a single polymer network can be provided by cross-linking PLA-PEG, PLA-PEG-PLA, PEG-PLA-PEG, PLGA-PEG, PLGA-PEG-PLGA, PEG-PLGA-PEG, polyfumarate-PEG, polyfumarate-PEG-polyfumarate, or PEG-polyfumarate-PEG block copolymers.

The compositions can also be cross-linked to form at least a portion of a polymeric matrix that comprises a semi-interpenetrating network. In these variations, the cross-linked PCL-PEG-PCL polymer network is infused or entangled with another linear or branched polymer. For example, the linear or branched polymer can be selected to have short or long chains to impart increased tensile strength and/or elasticity to the polymer matrix. The cross-linked polymer network can be selected to adjust the degradation of the polymer matrix in the body. For example, cross-linked PCL-PEG-PCL can be infused with long-chain collagen (see Example 3). In other variations, cross-linked PCL-PEG-PCL can be infused with gelatin, high molecular weight hyaluronic acid (HA), alginate or chitosan. For example, chitosan having a molecular weight from about 30 kDa to about 2000 kDa can be used, e.g., about 100 kDa to about 1000 kDa, or about 200 kDa to about 700 kDa. Alginate having a molecular weight from about 30 kDa to about 300 kDa can be used, e.g., about 50 kDa to about 150 kDa. HA having a molecular weight of about 70 kDa to about 4000 kDa can be used, e.g., about 100 kDa to about 2000 kDa, or about 500 kDa to about 1000 kDa. In some variations of the compositions, cross-linked PCL-PEG-PCL may be infused or entangled with more than one linear or branched polymer.

A semi-interpenetrating network can be formed by providing a solution of a functionalized cross-linkable block copolymer and a linear or branched polymer that does not cross-link to any significant extent when the block copolymer is cross-linked. Thus, a cross-linked network of the block copolymer can be formed around the chains of linear or branched polymer. Such a variation is provided in Example 3, where PCL-PEG-PCL is infused with long chain collagen. In other variations, a semi-interpenetrating network can be formed by providing a prepolymer (e.g., monomers or oligomers) as a first polymer and polymerizing and cross-linking the first polymer in the presence of a second polymer that does not polymerize or cross-link to any significant extent. For example, a solution of NHS-terminated PCL, amine-terminated PEG (amine-terminated at 3 or 4 positions), and modified collagen can be mixed to form a semi-interpenetrating network having a cross-linked PCL-PEG-PCL network infused with collagen. In this case, the collagen can be modified so that its amine groups do not react with the NHS group on the PCL. Alternatively, a solution of an amine-terminated PCL, NHS-terminated PEG (NHS-terminated at 3 or 4 positions) and modified collagen can be mixed to form a semi-interpenetrating network having a cross-linked PCL-PEG-PCL network infused with collagen. Here also, the collagen can be modified so that its amine groups do not react with the NHS groups.

In other variations, the compositions can be cross-linked to form at least a portion of a polymeric matrix that comprises an interpenetrating network. In these variations, a first polymer network is intertwined with a second polymer network. The combination of polymer networks can be chosen to impart increased tensile strength and/or elasticity to the polymer matrix, and/or to tune an in vivo degradation rate of the polymeric matrix.

For example, PCL-PEG-PCL can form an interpenetrating network with collagen. Collagen having a molecular weight of about 100 kDa to about 700 kDa, e.g., about 200 kDa to about 500 kDa can be solubilized to allow aqueous solutions of about 1% to about 15% (by weight). The collagen can be methylated or succinylated for solubilization. For example, the collagen can be methylated as described as follows. One kilogram of calf skin can be placed in 2 liters of hydrochloric acid (HCl) solution having a pH of about 2.5. Collagen can be added to the acidic solution, and pepsin can be added as an enzyme, where the ratio of pepsin to collagen is about 1:400. The solution can be stirred for about 5 days at ambient temperature. The viscous, solubilized collagen can be filtered using cheesecloth. The pepsin can be deactivated by adjusting the pH of the solution containing the solubilized collagen to about 10 by dropwise addition of NaOH, allowing the solution to stand for about 24 hours at about 4° C., and then adjusting the pH of the solution to about 7 by dropwise addition of HCl. The resulting collagen can be collected by centrifuge and freeze dried. The collagen can be placed in dehydrated methanol containing 0.1N HCl for about a week in a sealed container at room temperature, and then filtered and dried under vacuum. The methylated collagen can be redissolved at a concentration of about 1.5% (w/v) in an aqueous HCl solution having pH of about 3. In some cases, solubilized collagen or collagen that has not been solubilized may be dissolved in an acidic solution, e.g., in an acetic acid solution. An aqueous solution can be prepared from the solubilized collagen and PCL-PEG-PCL-DA. The solubilized collagen can be present in an amount of about 2% (w/v), about 4% (w/v), about 6% (w/v), about 8% (w/v), about 10% (w/v), or about 12% (w/v). The PCL-PEG-PCL-DA can be present in an amount of about 10% (w/v), about 20% (w/v), about 30% (w/v), about 40% (w/v), about 50% (w/v), or about 60% (w/v). First, the collagen can be cross-linked by any suitable method. For example, the collagen can be cross-linked using a cross-linking agent such as water-soluble 1-ethyl-3-(3-dimethyl aminopropyl) carbodiimide hydrochloride (EDC), EDC combined with NHS, a poly(ethylene glycol) functionalized with NHS, glutaraldehyde, or genipin. After the cross-linked collagen network has been formed in the solution, the PCL-PEG-PCL-DA can then be cross-linked by UV irradiation. Alternatively, the solution can be irradiated with UV such that the PCL-PEG-DA can be cross-linked first to form a gel. The gel can then be swelled with a solution of a cross-linking agent for the collagen, e.g., a solution of EDC, EDC combined with NHS, glutaraldehyde or genipin.

In other variations of the compositions, PCL-PEG-PCL can form an interpenetrating network with gelatin, as described in Example 4. In these variations, an aqueous solution containing gelatin in an amount of about 2% (w/v), about 5% (w/v), about 10% (w/v), about 15% (w/v) or about 20% (w/v), and PCL-PEG-PCL-DA in an amount of about 10% (w/v), about 20% (w/v), about 30% (w/v), about 40% (w/v), about 50% (w/v), about 60% (w/v), or about 70% (w/v). The molecular weight of the gelatin can be about 10 kDa to about 300 kDa, e.g., about 20 kDa to about 250 kDa, or about 50 kDa to about 100 kDa. The gelatin can be cross-linked in solution first by any suitable method, e.g., by using a cross-linking agent such as glutaraldehyde. The PCL-PEG-PCL-DA can be subsequently cross-linked using UV irradiation to form the interpenetrating network. Alternatively, the solution can be UV-irradiated first to cross-link the PCL-PEG-PCL-DA to form a gel comprising the gelatin. The gelatin can be subsequently cross-linked by swelling the gel with a solution of a cross-linking agent, e.g., glutaraldehyde, to form the interpenetrating network.

The compositions can include still other variations of polymeric matrices comprising an interpenetrating network. For example, an interpenetrating network between PCL-PEG-PCL and hyaluronic acid, alginate, chitosan, or PEG can be used. If PEG is used as the second network, then the mechanism used to cross-link the PCL-PEG-PCL should be distinct from the mechanism used to cross-link the PEG. For example, photocrosslinkable acrylated PEG can be used where the PCL-PEG-PCL network is formed with amine-terminated PCL combined with PEG terminated with NHS at 3 or 4 positions. In still other variations, two PCL-PEG-PCL networks can form an interpenetrating network. In these variations, two distinct PCL-PEG-PCL triblock copolymers can be provided that allow for independent cross-linking. For example, one of the triblock copolymers can be acrylated such that it can be cross-linked with UV, whereas the second of the triblock copolymers can be formed by cross-linking an amine-terminated PCL-PEG-PCL with PEG terminated with NHS at 3 or 4 positions.

The compositions can be used to form polymeric matrices formed by cross-linking a mixture of two polymeric components to form a super polymer network. For example, in some variations, the first polymeric component can be functionalized PCL-PEG-PCL. The PCL-PEG-PCL can be functionalized in any suitable manner, e.g., by acrylate termination, amine termination, NHS-termination, or sulfhydril-termination. The second polymeric component can be collagen, gelatin, functionalized PEG (e.g., acrylate-terminated, NHS-terminated, amine-terminated or sulfhydril-terminated), or a functionalized PCL-PEG-PCL as described above for the first polymeric component. A solution of the first and second polymeric components can then be prepared and cross-linking can be carried out to form the polymer matrix. Depending on the mixture chosen, several cross-linking mechanisms can occur. For example, cross-linking can occur between NHS-terminated PCL-PEG-PCL and collagen, gelatin, amine-terminated PEG, or amine-terminated PCL-PEG-PCL. Further, UV polymerization can be used to crosslink acrylated PCL-PEG-PCL, acrylated PEG, collagen or gelatin. Both collagen and gelatin require long UV exposures, e.g., longer than about 15 minutes, or several hours, e.g., 4 hours or more, if this cross-linking method is chosen. Catalyst-initiated cross-linking can also be used in some variations. In these variations, the first polymeric component can be an amine-terminated PEG or amine-terminated PCL-PEG-PCL. The second polymeric component can be collagen or gelatin. The cross-linking can be initiated with EDC, glutaraldehyde, or genipin.

The polymer matrices derived from the compositions described herein have at least one property that will allow a thin film or shell of the matrix surrounding a portion of a heart chamber to reinforce a heart wall to prevent dilatation of the heart chamber and/or expansion of an infarct. Elasticity or elastic modulus and tensile strength (e.g., tensile strength at break or ultimate tensile strength) of the matrices are examples of properties to be considered. Elastic modulus refers to the linear slope of a portion of a stress/strain curve, i.e., Δstress/Δstrain over a defined strain range. Ultimate tensile strength refers to the maximum tensile stress a material can support before failing.

Further, in vivo degradation rates of the matrix may be adjusted so that the reinforcing function of the polymer matrix lasts for at least about one month, at least about two months, at least about three months, at least about four months, at least about five months, or at least about six months or more. As used herein, an “in vivo” property encompasses that property in a living body, or that property in an environment designed to simulate the environment of a living body, e.g., by measuring the property in phosphate buffered saline (PBS), e.g., Dulbecco's phosphate buffered saline (DPBS).

The in vivo elastic modulus of a thin film of a polymeric matrix derived from the PCL-PEG-PCL compositions described herein may be selected so as to match the elastic modulus of a heart wall at strains representative of the normal expansion and contraction during beating. However, the in vivo elastic modulus of the polymeric matrix film should exceed the elastic modulus of the heart wall at higher strains so as to reinforce the heart wall and prevent dilatation and/or infarct expansion. Thus, films of the polymeric matrix can have a nonlinear in vivo elastic modulus characterized as being higher at strains above about 20% than at strains below about 20%. The thickness of the thin film of polymeric matrix can be about 200 μm to about 1 mm, e.g., about 250 μm, or about 300 μm, or about 400 μm, or about 450 μm, or about 500 μm, or about 750 μm.

For example, a wall of a normal functional heart has an elastic modulus of about 3 kPa to about 200 kPa at about 0% to about 20% strain. Thus, a film of the polymeric matrix can have an in vivo elastic modulus that approximates that of normal heart wall at strains below about 20%, e.g., about 3 kPa to about 200 kPa, or about 3 kPa to about 100 kPa, or about 3 kPa to about 30 kPa. But at strains above about 20%, e.g., from about 20% to about 30% strain, or from about 20% to about 40% strain, or from about 20% to about 50% strain, or from about 20% to about 60% strain, a film of the polymeric matrix can have an in vivo elastic modulus of about 200 kPa to about 1.5 MPa or even higher, e.g., about 300 kPa, about 400 kPa, about 500 kPa, about 600 kPa, about 700 kPa, about 800 kPa, about 900 kPa, about 1 MPa, about 1.1 MPa, about 1.2 MPa, or about 1.3 MPa. Therefore, in some variations, a thin film of a polymeric matrix can have an in vivo elastic modulus of about 3 kPa to about 100 kPa, or about 3 kP to about 30 kPa at strains below about 20% and an in vivo elastic modulus of 200 kPa or greater at strains above about 20%. Films of the polymeric matrices can have an ultimate tensile strengths of about 200 kPa, or about 300 kPa, or about 400 kPa, or about 500 kPa, or about 600 kPa, or about 700 kPa, or about 800 kPa or even higher. Further, some films of the polymeric matrices will have a desired combination of elastic modulus and tensile strengths. For example, some matrices will have an elastic modulus of about 200 kPa or higher, e.g., about 700 kPa, or about 900 kPa, or about 1 MPa, or about 1.5 MPa at strains above 20%, e.g., at strains from about 20% to about 40%, and an ultimate tensile strength of about 200 kPa or higher, e.g., about 300 kPa, or about 400 kPa, or about 500 kPa or even higher. Thus, such films or shells applied circumferentially to at least a portion of a heart can have sufficient elasticity to accommodate the change in heart volume as it fills, but can provide an inwardly-directed (toward the interior of the heart) force to resist further expansion of the myocardial wall and thereby prevent the heart from dilating or infarcts from expanding.

As stated above, the polymeric matrices derived from the compositions can be designed to degrade over time at a desired rate. In some cases, the rate of degradation of a polymeric matrix can be measured by a decrease in in vivo elastic modulus and/or in vivo tensile strength. For example, films of a polymeric matrix derived from the compositions may exhibit an average rate of decrease in in vivo elastic modulus of about 0.05% per day, such that the in vivo elastic modulus is decreased by about 9% over about 6 months. In other variations, the in vivo elastic modulus may decrease by an average rate of about 0.01%, about 0.1%, about 0.2%, about 0.4%, about 0.6%, about 0.8%, or about 1%, or about 2% per day. In still other variations, the in vivo elastic modulus may be maintained as relatively constant until the polymeric structure degrades after about 4 weeks, about 6 weeks, about 2 months, about 4 months, about 6 months or even longer.

Methods

Methods for reinforcing a myocardial wall of a heart chamber are described. The methods include accessing a pericardial tissue or a pericardial space and applying a sufficient amount of a polymeric matrix to the pericardial tissue or pericardial space to prevent dilation of the heart chamber and/or expansion of an infarct. The polymeric matrix can be derived from a triblock copolymer having the formula (CL)ₗ-(EG)ₘ-(CL)ₙ. The methods can be used to prevent dilation of the left ventricle, e.g., to treat congestive or chronic heart failure, or to prevent congestive or chronic heart failure. For example, the methods can be used to prevent expansion of an infarct, or to induce reverse remodeling in patients with already dilated hearts.

In the methods described herein, the pericardial tissue or pericardial space can be accessed using any suitable technique. For example, some methods can include image guidance, including fluorescence-enhanced image guidance techniques. In other methods, thoracoscopy can be used. In still other methods, the pericardial tissue or pericardial space can be accessed via a heart chamber or via an atrial wall. The methods can also include any combination of suitable techniques, e.g., image guidance can be used in combination with another technique.

The methods include applying a polymeric matrix to the pericardial tissue or pericardial space. “Applying” as used herein is meant to encompass delivering the polymeric matrix directly to the pericardial tissue or pericardial space, as well as delivering one or more matrix precursors (e.g., one or more polymer compositions that has not been cross-linked, one or more polymer compositions that has been partially cross-linked, or multiple components, e.g., monomers or oligomers, that can be polymerized and cross-linked to form the matrix) to the pericardial tissue or pericardial space. Further, the polymeric matrix or matrix precursors can be delivered to the pericardial space or tissue as a powder or a fluid, e.g., as a gel, liquid, suspension, or solution. Then the powder or fluid can be processed in situ to form a solid film of the matrix. For example, a fluid can be cured, e.g., by cross-linking, gelation, heating and/or drying. Further, a powder can be processed to form a solid film, e.g., by gelation, heating, and/or cross-linking. The pericardial tissue can be the fibrous pericardium, the parietal pericardium, or the visceral pericardium. In some variations of the methods, more than one polymeric matrix can be applied to a single heart. In other variations, a polymeric matrix can be applied to more than one location within a heart, e.g., more than one pericardial tissue, or a pericardial space and a pericardial tissue. In still other variations, the methods can include applying more than one polymeric matrix to a given location. The methods can also include applying a polymeric matrix in combination with one or more alternative cardiac support devices, e.g., devices comprising passive supports applied to the exterior of the heart, or passive supports similar to the CorCap™ support, the MyoSplint™ support, or the Paracor™ support, or devices providing active support to the heart. For example, methods can include using the polymeric matrices described herein to temporarily augment cardiac support provided by another device.

The methods include applying the polymeric matrix in an amount sufficient to prevent a chamber of a heart from dilating and/or an infarct from expanding. A sufficient amount can be defined in terms of a thin film or shell made from the matrix. Such a film or shell can surround or jacket at least a portion of the heart, e.g., around the left ventricle of the heart. The film or jacket can be disposed on the exterior surface of a heart wall (i.e., on the fibrous pericardium), or on an interior surface (the parietal pericardium or visceral pericardium), or in the pericardial space. Thus, a sufficient amount of polymeric matrix to prevent a chamber from dilating and/or an infarct from expanding encompasses a film of the matrix having an average thickness of about 200 μm, or about 250 μm, or about 300 μm, or about 400 μm, or about 500 μm, or about 750 μm, or about 1 mm, and having a surface area of about 30%, or about 40%, or about 50%, or about 60%, or about 70%, or about 80% or even more of the surface area of the myocardial wall that surrounds that chamber. In some variations, the film of the polymeric matrix may not form a solid shell, e.g., the film may comprise one or more bands or an open web of polymeric matrix. These open structure variations or the matrices may provide more rapid in vivo degradation.

The polymeric matrix applied to the pericardial tissue or pericardial space can be derived from A-B-A triblock copolymers having the formula (CL)ₗ-(EG)ₘ-(CL)ₙ. In some variations of the methods, l and n will be 1 to 18. For example, l can be 2 or 3 and n can be 2 or 3. In other variations, l can be 3 or 4 and n can be 3 or 4. The molecular weight of the PEG block can be about 1000 Da to about 15000 Da, about 3000 Da to about 12000 Da, about 5000 Da to about 10000 Da, about 6000 Da to about 8000 Da, or about 6000 Da. Correspondingly, m can be about 20 to about 400, e.g., about 70, about 110, about 130, about 150, about 180, about 200, about 250, about 300, or about 350. In other variations, the polymeric matrix applied to the pericardial tissue or pericardial space can be derived from A-B diblock copolymers having the formula (CL)ₗ-(EG)ₘ, where l and m can be varied as for the triblock copolymers described above.

In some variations of the methods, the polymeric matrix that is applied to the pericardial tissue or space comprises a semi-interpenetrating network. The semi-interpenetrating network can comprise a cross-linked network of the PCL-PEG-PCL triblock copolymer that is infused or entangled with a second polymer. In these variations, the methods can include providing a solution of a PCL-PEG-PCL triblock copolymer and the second polymer, and cross-linking the triblock copolymer in solution to form the semi-interpenetrating network. The second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof. For example, in some of these variations, the second polymer is collagen.

Other variations of the methods can include applying a polymeric matrix comprising an interpenetrating network to the pericardial tissues or space. The interpenetrating network can comprise a first cross-linked network derived from a PCL-PEG-PCL triblock copolymer interpenetrated with a second cross-linked network derived from a second polymer. In these variations, the methods can include providing a solution of the PCL-PEG-PCL triblock copolymer and the second polymer and cross-linking either the triblock copolymer or the second polymer in solution to form the first network, and subsequently cross-linking the other to form the second network, hence to form the interpenetrating network. The second polymer can be selected from the group consisting of alginate, casein, chitosan, chitin, collagen, gelatin, hyaluronic acid, poly(ethylene glycol) and derivatives thereof, poly(caprolactone) and derivatives thereof, blends thereof, and copolymers thereof. For example, the second polymer can be collagen.

The polymeric matrix can be formed prior to application to the pericardial tissue or space. The polymeric matrix can be formed in situ, i.e., during or after delivering the triblock copolymer to the pericardial tissue or space. In some variations, a polymeric matrix can be formed by in situ chemical cross-linking. For example, an amine-terminated PCL-PEG-PCL triblock copolymer can be cross-linked with an NHS-functionalized PEG (e.g., functionalized at 3 or 4 positions). In other variations, a polymeric matrix can be formed by cross-linking PCL-PEG-PCL-DA in situ using a UV light source. Further, a solution containing PCL-PEG-PCL-DA and a second polymer, e.g., long-chain collagen, can be applied to pericardial tissue or space, and a semi-interpenetrating network formed on the pericardial tissue or in the pericardial space by irradiating the solution with UV light. The UV light dose can be adjusted to cross-link the PCL-PEG-PCL-DA, but to avoid significant cross-linking in the second polymer. In still other variations of the methods, a polymeric matrix comprising an interpenetrating network can be formed in situ by carrying out two sequential cross-linking steps on a solution containing PCL-PEG-PCL-DA and a second polymer, where one of polymers is cross-linked in the solution to form a first network, and the other of the polymers is subsequently cross-linked to form a second network intertwined with the first network.

The methods can include selecting the in vivo elastic modulus of a thin film of a polymeric matrix derived from the PCL-PEG-PCL compositions to be nonlinear such that it matches the elastic modulus of a heart wall at strains representative of the normal expansion and contraction during beating and cardiac filling, but exceeds the elastic modulus of the heart wall at higher strains so as to reinforce the heart wall and prevent dilatation and/or infarct expansion. The thickness of the thin film of polymeric matrix can be about 200 μm to about 1 mm, e.g., about 250 μm, or about 300 μm, or about 400 μm, or about 450 m, or about 500 μm, or about 750 μm. Thus, a film of the polymeric matrix can have an in vivo elastic modulus that approximates that of normal heart wall at strains below about 20%, e.g., about 3 kPa to about 200 kPa, or about 3 kPa to about 100 kPa, or about 3 kPa to about 30 kPa. However, at strains above about 20%, for example, from about 20% to about 30% strain, or from about 20% to about 40% strain, or from about 20% to about 50% strain, or from about 20% to about 60% strain, a film of the polymeric matrix can have an in vivo elastic modulus of about 200 kPa, about 300 kPa, about 400 kPa, about 500 kPa, about 600 kPa, about 700 kPa, about 800 kPa, about 900 kPa, about 1 MPa, about 1.1 MPa, about 1.2 MPa, about 1.3 MPa, or about 1.5 MPa or even higher. In some variations, a thin film of a polymeric matrix can have an in vivo elastic modulus of about 3 kPa to about 100 kPa, or about 3 kP to about 30 kPa below about 20% strain and an in vivo elastic modulus of 200 kPa or greater at strains above about 20%. Further, films of the polymeric matrices can have an ultimate tensile strengths of about 200 kPa, or about 300 kPa, or about 400 kPa, or about 500 kPa, or about 600 kPa, or about 700 kPa, or about 800 kPa or even higher. Further, some films of the polymeric matrices can have a desired combination of elastic modulus and tensile strengths. For example, some films of the matrices will have an elastic modulus of about 200 kPa or higher, e.g., about 700 kPa, about 800 kPa, about 900 kPa, about 1 MPa, about 1.1 MPa, or about 1.5 MPa, or even higher at strains higher than about 20%, e.g., at about 20% to about 40% strain, and an ultimate tensile strength of about 200 kPa or higher, e.g., about 300 kPa, or about 400 kPa, or about 500 kPa, or even higher.

In some methods, the polymeric matrix disposed on the pericardial tissue or in the pericardial space is capable of reinforcing a myocardial wall for at least about 2 months, or at least about 4 months, or at least about 6 months, or even longer. In some variations of the methods, a biodegradable component of the polymeric matrix can be selected to have a desired in vivo degradation rate, which can be measured by a decrease in in vivo elastic modulus and/or in vivo tensile strength at break. For example, films of a polymeric matrix used in the methods may exhibit an average rate of decrease in in vivo elastic modulus of about 0.01%, 0.05%, about 0.1%, about 0.2%, about 0.4%, about 0.6%, about 0.8%, about 1%, or about 2% per day. In other variations of the methods, the elastic modulus can be maintained as approximately constant for about 4 weeks, about 2 months, about 4 months, about 6 months, or until the polymeric matrix dissolves or disintegrates.

The methods can include applying the polymer on or between one or more pericardial tissues using minimally invasive techniques. In some variations, the polymer can be delivered during a thoracoscopic procedure. During such a procedure, a conduit such as a needle or catheter is percutaneously placed through the chest wall. Conduit sizes can range from a size 4 French to a size 24 French. Once access has been obtained, the polymer matrix or a precursor to the polymer matrix is delivered into the pericardial space or to the surface of a pericardial tissue. The delivery of the polymer may be achieved by a variety of mechanisms, including a syringe, hand pump, automatic pump, or manual application as with a brush, applicator, or the like.

The amount of polymer that can be delivered can be about 10 cc to about 250 cc, e.g., about 25 cc to about 100 cc, or about 25 cc to about 50 cc. When the polymer is applied to an exterior surface of the heart, e.g., to the fibrous pericardium, higher volumes may be used, e.g., about 100 cc to about 500 cc, or about 150 cc to about 300 cc. The viscosity of the polymer in a fluid form can be between about 0.001 and 25 Pa-sec, e.g., about 1 to about 15 Pa-sec. Excess polymer may be removed by applying suction to the conduit, or by hand removal, e.g., by wiping away excess polymer. Depending on whether any cross-linking or curing is to occur in situ, the instrument that initiates cross-linking (e.g., a UV light source or a local heat source) may also be delivered to the pericardial space via the same or separate access point, before, during, and/or after the delivery of the polymer. A UV light source can be delivered via a fiber optic. In some cases, the access point can be sealed with the polymeric matrix that is being applied.

In further variations of the methods, a conduit, e.g., a catheter, may be used to access the pericardial space after advancement through the arterial or venous systems. For example, the methods can include advancing the conduit from the femoral vein to the right atrium, and then into the pericardial space through the atrial wall. The same conduit may be advanced into the pericardium by piercing any intrapericardial structures including, but not limited to coronary vessels, atria, ventricles, superior or inferior vena cava, pulmonary arteries, pulmonary veins, or aorta. Once access has been obtained, the polymer can be applied. As previously described, the beating of the heart can distribute the polymer within the pericardial space. In some cases, the application of the polymeric matrix, e.g., delivering a composition and/or performing an in situ process such as cross-linking can be coordinated, e.g., gated or synchronized, with the beating of the heart. In some cases, it may be desired to cross-link while cycle of the heart is in end-diastole. The same polymeric matrix, when cross-linked, may be used to seal a transpericardial or transmyocardial access point.

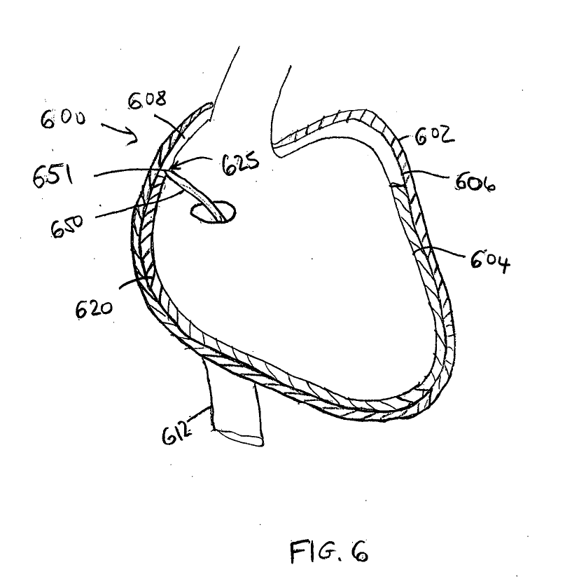

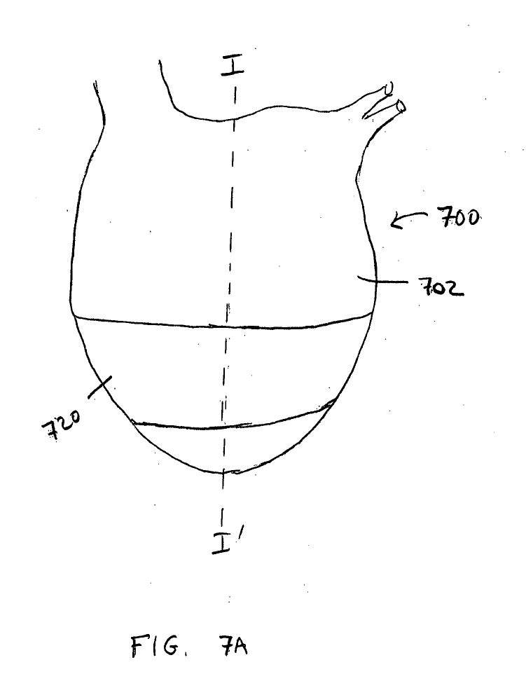

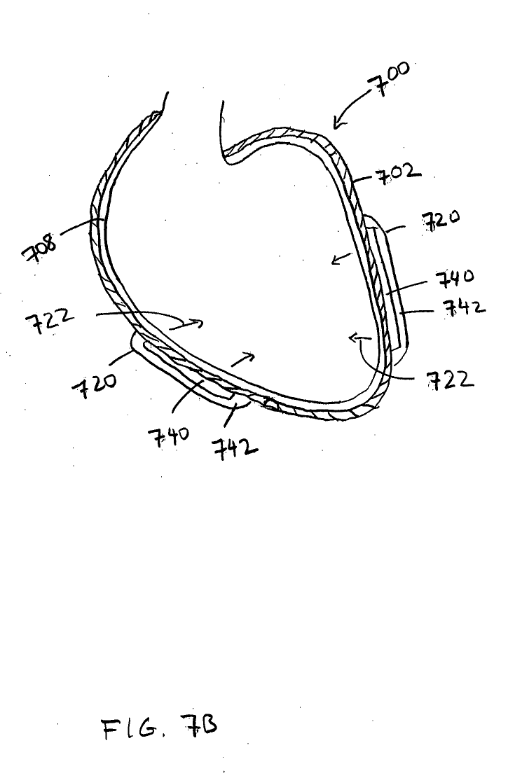

FIG. 6 illustrates a method in which a polymeric matrix is applied via a catheter into a femoral vein into the right atrium, and through an atrial wall into the pericardial space. Heart 600 has fibrous pericardium 602, visceral pericardium 604, parietal pericardium 606, and pericardial space 608. Catheter 650 enters the right atrium via a femoral vein (not shown) and pierces visceral pericardium 604 to access pericardial space 608 at position 625. A polymeric matrix or a polymeric matrix precursor 620 flows from catheter 650, e.g., driven by a pump or syringe (not shown), to fill a substantial portion (e.g., about 30%, about 40%, about 50%, about 60%, about 70% or more) of the volume of the pericardial space. A UV source, a local heat source, a cross-linking agent, or other cross-linking initiator may be provided to form the polymeric matrix within the pericardial space. For example, catheter 650 can include a fiber optic UV light source or a local heating element proximate to catheter tip 651. Alternatively, a separate catheter (not shown), or a separate channel within catheter 650 can be used to deliver a cross-linking initiator or a second component that can be used to form the polymeric matrix, e.g., a component that can cross-link with a precursor to the polymeric matrix. If a second catheter is used, it can access the pericardial space at the same or different access point than the first catheter. Access points, e.g., access point 625, can be sealed with the polymeric matrix after the delivery catheter is removed.

Some methods for reinforcing at least a portion of a wall of a heart chamber comprise accessing a pericardial tissue on the exterior of the heart, and applying a polymeric matrix in sufficient amount to the pericardial tissue to prevent dilation of the chamber or expansion of an infarct. For example, the PCL-PEG-PCL polymeric matrices described herein can be applied to the fibrous pericardium on the exterior of the heart. In these methods, the polymeric matrix can be applied by manually coating the heart, e.g., by brushing on the polymeric composition and cross-linking in situ to form a polymeric matrix. Polymeric matrices applied to the exterior of the heart need not form a solid shell, e.g., one or more bands around a portion of a heart may be formed, or a shell having an open web structure may be applied. Such variations may allow the polymeric matrix shell to degrade more rapidly.

Additional methods for reinforcing at least a portion of a myocardial wall of a heart chamber are described. These methods comprise accessing a pericardial tissue, applying a first layer of a first polymer to the pericardial tissue as a primer layer, and adhering a second layer of a second polymer to the first layer to form a polymeric matrix in sufficient amount to prevent dilatation of the chamber and/or expansion of an infarct. A sufficient amount of polymeric matrix to prevent a chamber from dilating and/or an infarct from expanding encompasses a film of the matrix having an average thickness of about 200 μm, or about 250 μm, or about 300 μm, or about 400 μm, or about 500 μm, or about 750 μm, or about 1 mm, and having a surface area of about 30%, or about 40%, or about 50%, or about 60%, or about 70%, or about 80% or even more of the surface area of the myocardial wall that surrounds that chamber. The pericardial tissue can be the fibrous pericardium, the parietal pericardium, and/or the visceral pericardium. The methods can include percutaneously inserting a first conduit to access the pericardial tissue, delivering the first polymer via the conduit to the tissue and forming the first layer thereon, and delivering the second polymer to the first layer via the first conduit or via a second conduit.

The first polymer is capable of forming a mechanical or chemical bond to the pericardial tissue. Any suitable biocompatible polymer may be used. The bond between the first polymer and the tissue may be temporary or permanent in nature. For example, a biodegradable polymer may be used to form the first polymer layer. For example, a polymer capable of adhering to tissue, e.g., a cyanoacrylate such as 2-octyl-cyanoacrylate, or collagen, may be used. In other variations, the first polymer can be a poly(caprolactone) or a copolymer thereof, e.g., a diblock or triblock copolymer comprising PCL blocks and PEG blocks. The first polymer can be applied to the pericardial tissue by delivering the first polymer or a precursor to the first polymer as a powder or as a fluid, e.g., a liquid, gel, suspension, or solution, to the pericardial tissue and processing the powder or fluid to form a solid film. For example, the fluid can be cured to form the solid film by cross-linking, gelation, heating, and/or drying. If the first polymer is delivered as a powder, it can be processed by heating, gelation, and/or cross-linking form a solid film. The average thickness of the first layer can be about 20 μm, 50 μm, about 100 μm, about 150 μm, about 200 μm, about 300 μm, or about 500 μm. The first layer need not be contiguous. The second layer may contact tissue directly in some variations, e.g., through openings in the first layer or at boundaries of the first layer.

The second polymer can also be capable of forming a mechanical or chemical bond with the tissue. The second polymer can comprise a triblock copolymer having the formula (CL)ₗ-(EG)ₘ-(CL)ₙ. In some variations, n and l can be 1 to 18. In other variations, n can be 2, 3 or 4 and l can be 2, 3 or 4. In still other variations, the molecular weight of the PEG block will be about 1000 Da to about 15000 Da, about 3000 Da to about 12000 Da, about 5000 Da to about 10000 Da, or about 6000 Da to about 8000 Da. In some variations, the second polymer can comprise a diblock PCL-PEG copolymer having the formula (CL)ₗ-(EG)ₘ, where l and m can be varied as for the triblock copolymers described above. The second layer can comprise a semi-interpenetrating network or an interpenetrating network between the second polymer, e.g., a PCL-PEG-PCL triblock copolymer, or a PCL-PEG diblock copolymer, and a third polymer. For example, a first cross-linked network of the second polymer can be infused or entangled with the third polymer to form a semi-interpenetrating network. In variations including a third polymer, e.g., as part of a semi-interpenetrating network or interpenetrating network, the third polymer can be capable of forming a mechanical or chemical bond to the tissue.

Alternatively, the second layer can comprise a first cross-linked network of the second polymer, e.g., a PCL-PEG-PCL triblock copolymer or a PCL-PEG diblock copolymer, that can be interlaced with a second cross-linked network of the third polymer. In these cases, the third polymer can comprise alginate, chitosan, casein, chitin, collagen, gelatin, hyaluronic acid, poly(ethylene glycol) and derivatives thereof, poly(caprolactone) and derivatives thereof, blends thereof, or copolymers thereof. In these variations, the second and/or third polymer may also be capable of forming a chemical or mechanical bond to the pericardial tissue.

The second layer can be adhered to the first layer with a cross-linking reaction between the first and second polymers. The second polymer can be functionalized to cross-link with the first polymer. For example, if collagen is used as the first layer, then PCL-PEG-PCL functionalized with NHS can be used as the second layer such that the NHS provides cross-linking between the collagen and triblock copolymer. The cross-linking between the first and second polymers results in a chemically-bonded laminate. In other variations, the second polymer or a precursor of the second polymer can be delivered as a powder or fluid, e.g., a liquid, gel, suspension, or solution, to the first layer and the powder or fluid can be processed in situ to form a solid laminate comprising the first and second polymers. A fluid form of the second polymer can be cured in situ by cross-linking, gelation, heating and/or drying. A powder form of the second polymer can be processed by gelation, heating, and/or cross-linking.

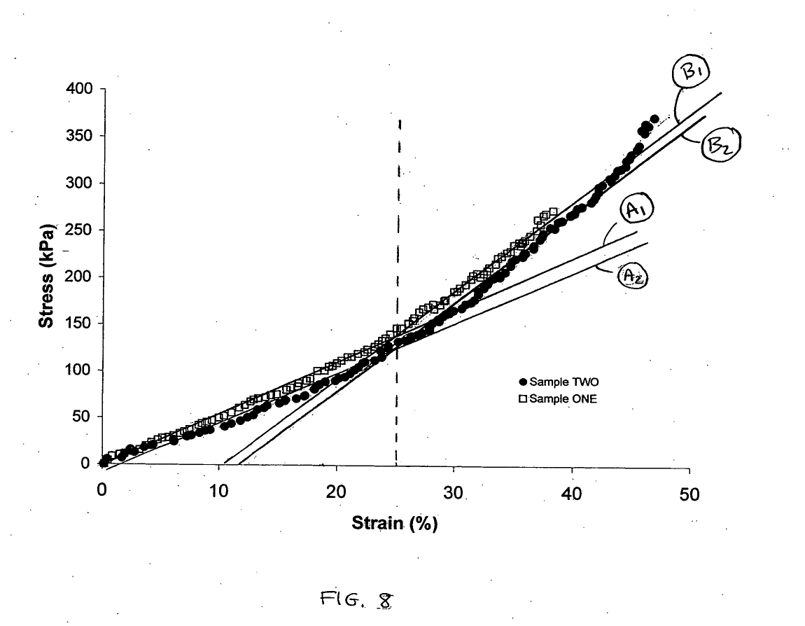

FIGS. 7A-7B illustrate a variation of a polymeric matrix comprising a two-layer laminate applied to the exterior of the heart. FIG. 7B shows a cross-sectional view along line I-I′ of FIG. 7A. Heart 700 has fibrous pericardium 702 and pericardial space 708. Polymeric matrix wall reinforcement 720 is applied as a band around a portion of heart 700 to apply an inward reinforcing force indicated by arrows 722. Reinforcement 720 comprises a two-layer laminate comprising an inner polymeric layer 740 that binds (mechanically or chemically) to the surface of fibrous pericardium 702 and an outer polymeric layer 742 adhered to layer 740.

Systems

Systems for reinforcing at least a portion of a myocardial wall to prevent dilatation of a chamber of a heart and/or expansion of an infarct are provided herein. The systems comprise a polymer composition adapted to form a polymeric matrix. The systems also comprise a first conduit configured to access a pericardial space or a pericardial tissue and to deliver the polymer to the pericardial space or tissue. As used herein, “delivering the polymer” encompasses delivering the polymeric matrix or delivering a polymer composition that is a precursor to the polymer matrix.

In some variations, the composition can comprise a triblock copolymer having the formula (CL)ₗ-(EG)ₘ-(CL)ₙ. For example, n and l can be 1 to 18. In other variations, n can be 2, 3 or 4 and l can be 2, 3 or 4. In still other variations, the molecular weight of the PEG block can be about 1000 Da to about 15000 Da, about 3000 Da to about 12000 Da, about 5000 Da to about 10000 Da, or about 6000 Da to about 8000 Da. In some variations of the systems, the compositions can comprise A-B diblock copolymers having the formula (CL)ₗ-(EG)ₘ, where l and m can be varied as for the triblock copolymers described above.

Some variations of the systems can comprise a second polymer, e.g., a second polymer selected from the group consisting of alginate, casein, chitin, chitosan, collagen, gelatin, hyaluronic acid, poly(ethylene glycol) and derivatives thereof, poly(caprolactone) and derivatives thereof, blends thereof, and copolymers thereof. For example, some systems can include collagen as a second polymer. In these systems, a PCL-PEG-PCL triblock copolymer can be adapted to form a polymeric matrix comprising a cross-linked network of the PCL-PEG-PCL triblock copolymer infused or entangled with the second polymer. In other systems including a second polymer, a PCL-PEG-PCL triblock copolymer can be adapted to form a polymeric matrix comprising an interpenetrating network comprising a first cross-linked network derived from the PCL-PEG-PCL triblock copolymer, and a second cross-linked network derived from the second polymer.

In further variations of the systems, a conduit, e.g., a catheter, may be adapted to access the pericardial space after advancement through the arterial or venous systems. For example, the conduit may be adapted to be advanced from the femoral vein to the right atrium, and then into the pericardial space. The same conduit may be adapted to be advanced into the pericardium by piercing any intrapericardial structures including, but not limited to coronary sinus, atria, ventricles, superior or inferior vena cava, pulmonary arteries, pulmonary veins, or aorta. Some conduits can include more than one separate channel. Systems can include a second conduit or even more conduits configured to access the pericardial space or a pericardial tissue. For example, in systems comprising a second polymer, a second conduit can be used to deliver the second polymer to the pericardial tissue or pericardial space. In other systems, a second conduit or a separate channel within the first conduit can be used to deliver a component that can cross-link with the polymer composition to form the polymeric matrix.

In other variations of the systems, the conduit for delivering the polymer composition may have a balloon at its tip. Before delivering the polymer, a thin flat balloon can be deployed percutaneously. The balloon can wrap around portions of the heart or around the entire heart. The balloon can be typically thin enough so that the hemodynamics of the heart are not significantly affected. After balloon deployment, the polymer can be delivered through small openings or holes in one or both sides of the balloon. The holes may be placed in any desired arrangement or pattern. For example, the holes may be configured to only deliver the polymer composition to the visceral pericardium. The balloon may be made from various materials, including, but not limited to, silicone, poly(isobutene), and polyurethane. The balloon may also be made of a biodegradable material so as to allow the operator to leave it in place after the application of the polymer to degrade over a desired time period. The balloon may also be devoid of openings or hole and the polymer composition applied in the balloon can remain in the balloon. The balloon can then degrade within the body.

Some variations of the systems can include an initiator to cause formation of at least a portion of the polymeric matrix. For example, the initiator can be capable of causing the generation of free radicals to facilitate cross-linking. Examples of suitable initiators may include: a light source, a radiation source (e.g., electron beam), a solution having a desired pH or ionic concentration, a heating device, a chemical cross-linker, a photoinitiator, or a combination thereof. For example, in some systems the initiator comprises a UV light source having a wavelength in the range of about 200 nm to about 400 nm.

The polymer compositions, methods and systems described here may be used to treat congestive or chronic heart failure of any etiology and any stage. For example, they may be used to treat newly diagnosed or symptomatic congestive or chronic heart failure due to myocardial infarction, myocardial ischemia, valvular dysfunction, hypertension, peripartum cardiomyopathy, connective tissue disease, chemotherapy-induced cardiomyopathy, substance abuse, diabetes, rheumatic disease, viral myocarditis, or unknown causes (idiopathic). They may also be used to prevent the development of congestive or chronic heart failure in patients at risk for its development, such as those with an acute myocardial infarction, a prior myocardial infarction, viral infection, valvular dysfunction, obesity, new onset of hypertension, connective tissue disease, cardiotoxic chemotherapy, substance abuse, and old age.

### EXAMPLES

### Example 1

### Synthesis of Acrylated Triblock Copolymer PCL-PEG-PCL-DA

A PCL-PEG-PCL triblock copolymer was synthesized as follows. ε-caprolactone (available from Aldrich) was purified by vacuum distillation over calcium hydride. 10 g of PEG (molecular weight 6000, available from Aldrich) was dried by azeotropic distillation using approximately 200 ml of dry toluene in a 500 ml flask. 3.4 g of the purified ε-caprolactone and 0.1% (w/v) stannous octoate (available from Spectrum Chemical) were added to the PEG/toluene solution under nitrogen atmosphere. The mixture, still under nitrogen atmosphere, was stirred for 24 hours at 115° C. by heating with a silicone oil bath set to 150° C. The mixture was then cooled to room temperature, and refrigerated at 0° C. to 4° C. for at least 24 hours. After refrigeration for at least 24 hours, the polymer was precipitated from solution by slow dropwise addition of 300 ml of anhydrous hexane. The precipitated PCL-PEG-PCL polymer was isolated from solution by filtering under vacuum using a Buchner filter with two filter papers saturated with hexane. The isolated PCL-PEG-PCL was placed in a dessicator under vacuum for at least 48 hours. The resulting PCL-PEG-PCL triblock copolymer was verified using NMR spectroscopy, and had a PEG block molecular weight of about 6000 Da, and 2 CL units in each of the PCL blocks, with an overall yield of 54% to 86%.

The PCL-PEG-PCL was acrylated according to the following procedure. 7.31 g of PCL-PEG-PCL in powder form was dissolved in approximately 100 cc of anhydrous tetrahydrofuran. 2.0 ml of acryloyl chloride (available from Aldrich) was added dropwise to the PCL-PEG-PCL solution over a period of 30 minutes while continuously stirring with a magnetic stirrer. The mixture was refluxed at 50° C. for 5 hours under a nitrogen atmosphere, cooled to room temperature, and then the reflux procedure was repeated. After the reflux procedure, 300 ml of hexane was added dropwise to precipitate PCL-PEG-PCL-DA. The precipitated PCL-PEG-PCL-DA was filtered using a glass filter and washed three times with 30 cc hexane. The PCL-PEG-PCL-DA was dried in a Petri dish in a desiccator under vacuum for at least 48 hours. The product was further purified by re-dissolving in 80 cc of tetrahydrofuran and re-precipitating with the addition of 150 cc hexane. The resulting white precipitate was filtered, washed and dried under vacuum in a desiccator. The PCL-PEG-PCL-DA was characterized using proton NMR. The yield for the acrylation step was 71% to 91%.

### Example 2

### Formation of Polymer Network

(PCL)₂-PEG-(PCL)₂-DA as synthesized in Example 1 was dissolved in water at 50% (w/v). A photoinitiator (2-hydroxy-2-methylpropiophenone) at about 1% (w/v) was added to solution. The solution was then placed in a transparent container formed by two glass slides spaced apart by a 500 μm spacer. The solution was then irradiated with a UV light source (75 W Xenon arc lamp, Oriel model 6251NS, about 1.0 mW/cm²) for about 1 to 10 minutes to form a cross-linked network from the (PCL)₂-PEG-(PCL)₂-DA copolymer.

### Example 3

### Formation of Semi-Interpenetrating Polymer Network

A thin film of a semi-interpenetrating network of (PCL)₂-PEG-(PCL)₂ and collagen was prepared. An aqueous solution of (PCL)₂-PEG-(PCL)₂-DA copolymer (synthesized as in Example 1) at 50% (w/v), long-chain collagen (molecular weight about 300 kDa, available from Becton-Dickinson) at 1% (w/v) (solubilized as described above), and 1% (w/v) photoinitiator (2-hydroxy-2-methylpropriophenone) was deposited in a transparent container made from two glass slides spaced apart by a 500 μm spacer. The solution was then irradiated with a UV light source as in Example 2 for about 1 to 10 minutes to form a semi-interpenetrating polymer network comprising a cross-linked (PCL)₂-PEG-(PCL)₂ network infused or entangled with long-chain collagen.

### Example 4

### Formation of Interpenetrating Network

An interpenetrating network between (PCL)₂-PEG-(PCL)₂ and gelatin was formed as follows. (PCL)₂-PEG-(PCL)₂-DA was synthesized as in Example 1. An aqueous solution of two percent type B gelatin from bovine skin (molecular weight about 40 kDa to about 50 kDa, available from Sigma) gelatin was prepared. An aqueous solution of 50% (w/v) of (PCL)₂-PEG-(PCL)₂-DA in the 2% gelatin solution was created. A photoinitiator (2-hydroxy-2-methylpropiophenone) was added at 1% (w/v) and the hydrogel solution was then placed in transparent container formed with two glass slides separated by a 500 μm spacer and cross-linked as described in Example 2. The cross-linked hydrogel was then immersed in a 0.5 M solution of glutaraldehyde (Sigma) in PBS for 24 hours to allow cross-linking of the gelatin.

### Example 5

### Polymer Matrix Characterization Studies of a Cross-Linked Network

Polymeric matrices were characterized by performing a uniaxial tensile test using an Instron™ Model 5844 single column testing system, equipped with a 10N load cell, and a BioPuls™ bath, and submersible pneumatic grips. Bluehill2® software was used to operate the Instron™ tester. The method generally followed was ASTM Standard D638-03, Standard Test Method for Tensile Properties of Plastics. Thin films of exemplary polymeric matrices were soaked in DPBS in a Petri dish for at least 24 hours to simulate in vivo conditions. The films were stamped to form dumbbell shaped samples, having length of 9.5 mm, width of 3.18 mm, and a thickness ranging from 250 μm to 750 μm. The thickness of the samples was measured by placing the samples between two glass slides and using a caliper to measure the thickness of all three layers and subtracting the glass slide thicknesses so that the sample was not compressed during thickness measurement. A sample was clamped between grips, and submerged into the BioPuls™ bath (filled with 3.0 L PBS 7.40 buffer). The sample was stretched uniaxially at 15 mm/min until rupture or slipping out of grips. By stretching the samples to yield, and then rupture, a stress/strain curve for each sample was generated according to standard techniques.

The stress/strain curves were analyzed by fitting one or more straight lines through different areas of the stress/strain curve, as shown in FIG. 8. In FIG. 8, stress/strain curves for two 500 μm samples as prepared in Example 2 are shown. The samples were immersed for 24 hours in PBS buffer prior to testing. As indicated by solid lines A₁ and A₂, the samples have an elastic modulus of about 500-550 kPa in strain region below about 25% (25% strain is indicated by a dashed line in the Figure). Above strain regions of about 25%, the samples have an elastic modulus of about 940 kPa to about 1100 kPa, as shown by solid lines B₁ and B₂. The maximum stress withstood before failure (ultimate tensile strength) by samples one and two was 274 kPa and 373 kPa, respectively.

### Example 6

### Polymer Matrix Characterization Studies of a Semi-Interpenetrating Network

A sample was prepared as in Example 3, except that the aqueous solution contained 1% (w/v) chitosan instead of 10% collagen. The sample was immersed in PBS for 24 hours prior to testing. The mechanical properties of a 500 μm film of the sample were measured as described in Example 5. The in vivo elastic modulus for this sample was 465 kPa below 22% strain and 862 kPa above 22% strain. The maximum stress withstood by this sample before failure (ultimate tensile strength) was 217 kPa.

### Example 7

### Degradation Testing of a PCL-PEG-PCL Network

A set of 500 μm thick cross-linked PCL-PEG-PCL samples was prepared as in Example 2. A first sample of the set was immersed for 24 hours in PBS, a second sample was immersed in PBS for 23 days, and a third sample was immersed in PBS for 71 days. Mechanical testing on all three samples was done after their respective immersion periods as described in Example 5. Results are shown in Table 1 below. Modulus A refers to the slope of the stress/strain curve at low strains (e.g., below 20-25%) and Modulus B refers to the slope of the stress/strain curve at higher strains (above 20-25%). In this case, Modulus B decreased on average by about 0.35% over the entire 71 days.

TABLE 1
Degradation Study

| | | Max | | | |
|---|---|---|---|---|---|
| | Immersion | stress | Modulus A | Strain at which | Modulus B |
| Sample | time | (kPa) | (kPa) | slope changes | (kPa) |
| 1 | 24 hours | 274 | 550 | 25% | 940 |
| 2 | 23 days | 169 | 518 | 22% | 741 |
| 3 | 71 days | 178 | 502 | 22% | 710 |

### Example 8

### Animal Protocol

The following animal protocol is planned for evaluating the application of the polymeric matrices described herein to a heart to prevent progressive dilation and remodeling of the heart after an acute myocardial infarction. The animals will be chronic heart failure animal models. All animals will receive humane care in compliance with the Principles of Laboratory Animal Care as promulgated by the National Society for Medical Research and the Guide for Care and Use of Laboratory Animals prepared by the National Academy of Sciences and published by the National Institutes of Health (NIH Publication 85-23).

Dorsett sheep will undergo an anterior myocardial infarction. The mechanical operation and physical shape of the hearts following the myocardial infarction will be studied before and after placing a polymeric matrix around the heart to reinforce a heart wall. Prior to studying chronic effects, an acute study will be carried out to evaluate the hemodynamic effects and characteristics of a polymeric reinforcing device on normal hearts under varied loading conditions. After the acute study, a chronic study will assess the long-term effects of the application of various polymeric matrices in the hearts of four sheep. These long-term studies will include monitoring the effect of the polymeric matrix on heart function and ventricular remodeling after myocardial infarction.

Four Dorsett hybrid male sheep will be premedicated with ketamine (25 mg/kg) for venous and arterial catheter placement. The sheep will be anesthetized using thiopental sodium (6.8 mg/kg IV) and kept under anesthesia using inhalational isoflurane (1-2.5%) in supplemental oxygen. Single doses of antibiotics (1 g IV cefazolin sodium and 80 mg IV gentamicin sulfate) will be administered preoperatively and antibiotics will be continued during the early postoperative period. The sheep will be given an acute myocardial infarction according to the infarct model described by Gorman et al., Infarct Size and Location Determine Development of Mitral Regurgitation in the Sheep Model, J. Thorac. Cardiovasc. Surg. 1998; 115(3); 615-22. A left thorocotomy will be performed to identify vessels to be ligated, i.e., any diagonal or obtuse margin vessel that supplies the anterior portion of the myocardium, excluding the left anterior descending coronary artery. A pneumatic occluder (available from In Vivo Metric Systems, Healdsbug, Calif.) will be placed around the identified vessels to create a large anterior wall infarction.

A micromanometer-tipped pressure catheter and conductance catheter (Millar SPC-500, available from Millar Instruments, Houston, Tex.) will be inserted into the left ventricle apex of the animals to allow measurement of pressure and the generation of pressure-volume curves for each sheep heart. All animals will receive IV magnesium (3 g), lidocaine (100 mg), and bretylium (50 mg) as post-infarction arrhythmia prophylaxis before baseline data acquisition. After a 5-minute wait following administration of the arrhythmia prophylaxis, the obtuse marginal and diagonal coronary arteries of the animals will be occluded for about 1 minute, and then ischemic data will be measured and recorded. Systolic blood pressure will be maintained at 80 mm Hg throughout the procedure using repeated bolus doses of phenylephrine.

Polymeric reinforcement devices will be placed on the hearts of all the study animals immediately following infarction. An incision will be made, the pericardium will be opened and a polymeric composition in a fluid form or powder form can be manually applied to the epicardial surface of the heart, e.g., using a brush, an applicator, or the like. The polymer composition will be chosen to chemically or mechanically bind to the tissue surface. The polymer composition will then be cured or processed (e.g., cross-linked) to form a polymeric matrix surrounding and supporting at least a portion of the heart wall. For example, a 50% (w/v) aqueous solution of PCL-PEG-PCL-DA, with each PCL block comprising 2-3 monomeric caprolactone units and the PEG block having a molecular weight of about 6000 Da to about 8000 Da will be applied to the epicardial surface of the heart. The area of the epicardial surface to be covered will be about 40 to about 50%, or even higher, depending on the amount of support desired. The PCL-PEG-PCL-DA will then be cross-linked by irradiation with a UV source to form a solid film forming a shell around at least a portion of the heart. In some of the experiments, more than one layer of polymer may be applied to build up a film of a desired thickness, e.g., about 250 μm to about 500 μm. In some experiments, the cross-linking will be synchronized to the beating of the heart by electrocardiographically gating the cross-linking to occur at a certain time in the cardiac cycle, e.g., at end-diastolic volume. After the matrix is applied to the heart, the incision will be closed in layers, and a chest tube placed to drain the chest and remove any pneumothorax. The chest tube will be removed once the animal is ambulatory.

In some of the experiments, a coating of solubilized collagen (molecular weight of about 30 kDa to about 300 kDa) will be applied to the tissue wall first as a primer. The collagen will mechanically and/or chemically bind to the tissue wall. Then PCL-PEG-PCL functionalized with NHS will be applied to the collagen layer and cross-linked in situ.

After the polymeric matrix is applied to the animal hearts, the vital signs, electrocardiogram, and hemodynamic status of the animals will be monitored for at least 24 hours postoperatively. Intravenous fluids, hemodynamic support, arrhythmia medications, and antibiotics will be continued in the immediate postoperative period as necessary. Once an animal is considered hemodynamically stable, it will be returned to the animal colony.

Four sets of measurements will be taken for each animal: at baseline; immediately after infarction, after application of the polymeric reinforcement device; and prior to sacrifice at 6 weeks. The measurements will include a pressure-volume analysis, echocardiography, and hemodynamics. The first three sets of measurements will be taken during the initial operative procedure as described above.

For the final set of measurements to be taken 6 weeks postoperatively, each animal will be sedated with ketamine (1-4 mg/kg IV), placed in the right lateral decubitus position, intubated, mechanically ventilated, and maintained with inhalational isoflurane (1-2.5%) in 100% oxygen. A micromanometer-tipped pressure transducer as described above, previously calibrated in a water bath, will be advanced into the left ventricle along the long axis of the left ventricular cavity via a carotid artery catheter using fluoroscopic guidance techniques. This transducer will enable left ventricle pressure measurements. To obtain volume measurements from the left ventricle, a 7F multielectrode dual-field conductance catheter (Webster Labs, Baldwin Park, Calif.) will be placed along the long axis of the left ventricular cavity through a sterile cutdown of the right femoral artery. Thereafter, 20 ml occlusion catheters (Applied Vascular, Laguna Hills, Calif.) will be placed at the junction of the superior and inferior venae cavae with the right atrium through the right jugular and right femoral veins. A balloon-tipped pulmonary artery catheter will then be placed through the left jugular vein. All incisions will be closed primarily.

Volume measurements will be obtained via the conductance catheter technique, as described by Kass et al., Determination of Left Ventricular End-Systolic Pressure-Volume Relationships by the Conductance (Volume) Catheter Technique, Circulation 1986; 73(3): 586-95. All hemodynamic signals and electrocardiographic tracings will be stored and processed with LabVIEW™ (National Instruments, Austin, Tex.) data collection and analysis software. All data points will be collected with the ventilator held at end-expiration. To determine the end-systolic and end-diastolic pressure-volume relationships and the preload-recruitable stroke work relationship (the slope of a linear plot of stroke work vs. end-diastolic volume), the 20 ml balloons in the venae cavae will be gradually and temporarily inflated to alter preload and measure left ventricle pressure and stroke volume with an echocardiographic aortic flow probe.

Subdiaphragmatic 2D echocardiographic images will be obtained using a sterile, midline laparotomy with a 5 MHz probe (Hewlett Packard 77020A). Left ventricular short-axis images at three levels (the tip and base of the papillary muscles and the apex) and two orthogonal long-axis views will be obtained. Left ventricular apical long-axis views will then be used to calculate the left ventricle cavity volumes by biplane Simpson's rule (Weyman, Principles and Practice of Echocardiography; 1994). Serial echocardiographic measurements will be made of the left ventricle cavity diameter at the tip of the papillary muscles and left ventricle long axis cavity length to calculate the left ventricle cavity shape, defined as the ratio of the short axis to the long axis. Left ventricular wall thickness will be measured from short axis images at the base of the papillary muscle, apex, infarct zone, and remote areas, at both end-diastole and end-systole. Myocardial infarct length will be measured as the length of left ventricle cavity perimeter that is either akinetic or dyskinetic. The percent of akinetic or dyskinetic length out of the total cavity perimeter will then be calculated (St. John Sutton et al., Quantitative Two-Dimensional Echocardiographic Measurements are Major Predictors of Adverse Cardiovascular Events after Acute Myocardial Infarction. The Protective Effects of Captopril, Circulation 1994; 89(1): 68-75).

After the follow up measurements at 6 weeks, the animals will be euthanized. While on anesthesia, conventional 3.0 mm perfusion balloon catheters will be placed in the proximal left anterior and left circumflex coronary arteries. The animals will be euthanized by administration of Pentothal Sodium (1 g IV) followed by an intravenous bolus of KCL (80 mEq) to depolarize and arrest the heart at end-diastole. The left ventricle pressure will be measured while central venous exsanguination is performed until it matches the measured in vivo left ventricle end-diastolic pressure. After these two pressure measurements are matched, the hearts will be fixed in situ by simultaneous injection of 300 ml of 5% buffered glutaraldehyde into each coronary catheter. The hearts will be excised and stored in 10% formalin for tissue histology and wall thickness measurements. Tissue viability will be determined by hematoxylin and eosin and/or triphenyl tretrazolium chloride staining. Collagen content will be determined using picrosirius red staining (Blom et al., Cardiac Support Device Modifies Left Ventricular Geometry and Myocardial Structure After Myocardial Infarction, Circulation 2005; 112(9): 1274-83).

All publications, patents, and patent applications cited herein are hereby incorporated by reference in their entirety for all purposes to the same extent as if each individual publication, patent, or patent application were specifically and individually indicated to be so incorporated by reference. Although the foregoing compositions, methods, and systems have been described in some detail by way of illustration and example for purposes of clarity of understanding, it will be readily apparent to those of ordinary skill in the art, in light of the description herein provided, that certain changes and modifications may be made thereto without departing from the spirit and scope of the appended claims.

1. A composition comprising a triblock copolymer having the formula (CL)ₗ-(EG)ₘ-(CL)ₙ, wherein:
CL is a caprolactone monomeric unit;
EG is an ethylene glycol monomeric unit;
l is an integer from 1 to 18;
n is an integer from 1 to 18;
m is an integer from 70 to 400; and
the triblock copolymer is functionalized with at least one crosslinkable group.

2. The composition of claim 1 wherein the crosslinkable group is an acrylate, an amine, a sulfhydril, or N-hydroxysuccinimide.

3. The composition of claim 1 wherein the triblock copolymer is terminated with a cross-linkable acrylate group.

4. The composition of claim 1 wherein m is 130 to 200.

5. The composition of claim 1 wherein l is 2 or 3 or 4 and n is 2 or 3 or 4.

6. The composition of claim 1 cross-linked to form at least a portion of a polymeric matrix.

7. The composition of claim 6 wherein the polymeric matrix has an in vivo elastic modulus of about 200 kPa or greater at strains of about 20% or higher.

8. The composition of claim 6 wherein the polymeric matrix has an in vivo ultimate tensile strength of about 200 kPa or greater.

9. The composition of claim 6 wherein the polymeric matrix comprises a semi-interpenetrating network comprising a first cross-linked network derived from the triblock copolymer and a second polymer infused into the first cross-linked network.

10. The composition of claim 6 wherein the polymeric matrix comprises an interpenetrating network comprising a first cross-linked network derived from the triblock copolymer and a second cross-linked network derived from a second polymer.

11. The composition of claim 6 wherein the triblock copolymer is functionalized with an amine, and the polymeric matrix is at least partially formed by cross-linking the functionalized triblock copolymer with a poly(ethylene glycol) functionalized with N-hydroxysuccinimide.

12. The composition of claim 1 adapted to be delivered by injection.

13. A method for reinforcing a wall of a heart chamber comprising:
accessing a pericardial tissue or a pericardial space; and
applying a sufficient amount of a polymeric matrix to the pericardial tissue or the pericardial space to prevent dilatation of the chamber or expansion of an infarct, wherein:
the polymeric matrix is derived from a triblock copolymer having the formula (CL)ₗ-(EG)ₘ-(CL)ₙ, and wherein:
CL is a caprolactone monomeric unit;
EG is an ethylene glycol monomeric unit; and
l, m, and n are integers.

14. The method of claim 13 wherein the pericardial tissue is selected from the group consisting of the fibrous pericardium, the parietal pericardium, and the visceral pericardium.

15. The method of claim 13 wherein the heart chamber is the left ventricle.

16. The method of claim 13 comprising accessing the pericardial tissue or pericardial space using thoracoscopy.

17. The method of claim 13 comprising accessing the pericardial tissue or pericardial space via a heart chamber.

18. The method of claim 13 comprising accessing the pericardial tissue or pericardial space via an atrial heart wall.

19. The method of claim 13 wherein the polymeric matrix comprises a semi-interpenetrating network comprising a cross-linked network of the triblock copolymer that is infused with a second polymer.

20. The method of claim 13 wherein the polymer matrix comprises an interpenetrating network comprising a first cross-linked network derived from the triblock copolymer interpenetrated with a second cross-linked network derived from a second polymer.

21. The method of claim 13 wherein the triblock copolymer is functionalized with an amine, and the polymeric matrix is at least partially formed by cross-linking the functionalized triblock copolymer with a poly(ethylene glycol) that is functionalized with N-hydroxysuccinimide.

22. The method of claim 13 wherein the polymeric matrix has an in vivo elastic modulus of about 200 kPa or greater at strains above about 20%.

23. The method of claim 13 wherein the polymeric matrix has an in vivo ultimate tensile strength of about 200 kPa or greater.

24. The method of claim 13 wherein the polymeric matrix is capable of reinforcing the wall of the heart chamber for at least about 2 months.

25. The method of claim 13 wherein at least a portion of the polymeric matrix is formed prior to application.

26. The method of claim 13 comprising:
percutaneously inserting a conduit to access the pericardial space or pericardial tissue; and
applying the polymeric matrix or a precursor form of the polymeric matrix to the pericardial tissue or pericardial space via the conduit.

27. The method of claim 13 wherein the conduit is inserted through a femoral vessel.

28. The method of claim 13 wherein at least a portion of the polymeric matrix is formed during or after delivering the triblock copolymer to the pericardial tissue or the pericardial space.

29. The method of claim 28 wherein at least a portion of the polymeric matrix is formed by UV irradiation of the triblock copolymer after the triblock copolymer has been delivered to the pericardial tissue or the pericardial space.

30. The method of claim 13, wherein the polymeric matrix is applied to the pericardial tissue or the pericardial space by:
delivering the polymeric matrix or a precursor to the polymeric matrix to the pericardial tissue or the pericardial space as a fluid or powder; and
processing the fluid or powder in situ to form a solid film.

31. The method of claim 13 for treating chronic heart failure or for preventing chronic heart failure.

32. The method of claim 13 for preventing dilatation of a heart chamber or preventing expansion of an infarct.

33. A method for reinforcing at least a portion of a wall of a heart chamber comprising:
accessing a pericardial tissue;
applying a first layer of a first polymer to the pericardial tissue; and
adhering a second layer of a second polymer layer to the first layer to form a polymeric matrix in sufficient amount to prevent dilatation of the chamber or expansion of an infarct.

34. The method of claim 33 wherein the tissue is selected from the group consisting of the fibrous pericardium, the parietal pericardium, and the visceral pericardium.

35. The method of claim 33 wherein at least one of the first and second polymer comprises a triblock copolymer having the formula (CL)ₗ-(EG)ₘ-(CL)ₙ, wherein:
CL is a caprolactone monomeric unit;
EG is an ethylene glycol monomeric unit; and
l, m, and n are integers.

36. The method of claim 33 wherein the first polymer is applied to the pericardial tissue by delivering the first polymer or a precursor of the first polymer as a fluid or powder to the pericardial tissue and processing the fluid or powder to form a solid film.

37. The method of claim 33 wherein second layer is adhered to the first layer by delivering the second polymer or a precursor of the second polymer as a fluid or powder to the first layer and processing the fluid or powder to form a solid laminate comprising the first and second polymers.

38. A system for reinforcing at least a portion of a wall of a heart chamber comprising:
a polymer composition adapted to form a polymeric matrix, the composition comprising a triblock copolymer having the formula (CL)ₗ-(EG)ₘ-(CL)ₙ, wherein CL is a caprolactone monomeric unit, EG is an ethylene glycol monomeric unit and l, m, and n are integers; and
a first conduit configured to access a pericardial space or a pericardial tissue and deliver the polymer composition or the polymeric matrix to the pericardial space or tissue.

39. The system of claim 38 comprising an initiator to cause formation of at least a portion of the polymeric matrix.

40. The system of claim 38 wherein the conduit comprises a balloon configured to deliver the polymer composition or the polymeric matrix to the pericardial space or tissue.
